SEQUENCE LISTING (19 sequences)

SEQ ID NO: 1 - DNA, 35 bp, Artificial Sequence
  chemically synthesized, BMF Forward primer
gcggcggctagcaactatcctttgcagacagacgg

SEQ ID NO: 2 - DNA, 36 bp, Artificial Sequence
  chemically synthesized, BMF Reverse primer
gcggcgaagcttaaaatacgcctgctcggggcatcc

SEQ ID NO: 3 - DNA, 38 bp, Artificial Sequence
  chemically synthesized, IL1beta Forward primer
gcggcggctagcgactgcttttctgaaatgatgcaagg

SEQ ID NO: 4 - DNA, 35 bp, Artificial Sequence
  chemically synthesized, IL1beta Reverse primer
gcggcgagatctggaagcttcttaggggaggggac

SEQ ID NO: 5 - DNA, 41 bp, Artificial Sequence
  chemically synthesized, IGFBP3 forward primer
gcggcggctagcgtctattggtggtcagtaagactgatagc

SEQ ID NO: 6 - DNA, 41 bp, Artificial Sequence
  chemically synthesized, IGFBP3 reverse primer
gcggcgaagcttgacgcctgcaaccggggcacgctgcttgg

SEQ ID NO: 7 - DNA, 39 bp, Artificial Sequence
  chemically synthesized, Lamp3 forward primer
gcggcggctagcgatcaacctccagccaaacttatcagg

SEQ ID NO: 8 - DNA, 41 bp, Artificial Sequence
  chemically synthesized, Lamp3 reverse primer
gcggcggatatcaacgaaaccacctgcttatgagaagcagc

SEQ ID NO: 9 - DNA, 23 bp, Artificial Sequence
  chemically synthesized, IL1beta forward rt-PCR primer
aatctgtacctgtcctgcgtgtt

SEQ ID NO: 10 - DNA, 26 bp, Artificial Sequence
  chemically synthesized, IL1beta reverse rt-PCR primer
tgggtaatttttgggatctacactct

SEQ ID NO: 11 - DNA, 21 bp, Artificial Sequence
  chemically synthesized, BMF forward rt-PCR primer
atggagccatctcagtgtgtg

SEQ ID NO: 12 - DNA, 20 bp, Artificial Sequence
  chemically synthesized, BMF reverse rt-PCR primer
ccccgttcctgttctcttct

SEQ ID NO: 13 - DNA, 22 bp, Artificial Sequence
  chemically synthesized, IGFBP3 forward rtPCR primer
gtccaagcgggagacagaatat

SEQ ID NO: 14 - DNA, 20 bp, Artificial Sequence
  chemically synthesized, IGFBP3 reverse rtPCR primer
cctgggactcagcacattga

SEQ ID NO: 15 - DNA, 3996 bp, Artificial Sequence
  chemically synthesized, BMF promoter sequence
aactatcctttgcagacagacggataacagataaagtccctttgatagggcaagtccggcaagggaaggacacccacgtcttgcagatccctggtctttcttacatatatgagctcttctggaagtagttaagggggtcaggcaccatgtgctcccccagcaagaccccaaccttacccataaatcagtttcccatctaacccctccctgggggttgagttacaaacctcagtaaccccagggcagcctgcctggtaattcctattattcagctgtcagctggttttttaaaaaccgccctttccagtgatccagttactggagttttagagccatttcttgtctcagagagaaatcctatagccaggtgagacaatggagagggctcctgcccgctttctacaggagctgggttttctctctggttgagagcagcctccccttaactctgtatccctccaagattgtcagggaaggccctcctccctatcagcctccgtgctgatgcctttctctggggacccttctcccagagccagcatcagttagcagaggagaaaacaaattgagacaaggtgcaggaagagaatgagagaaaggaaattctccaaaatagaggttgctgtgactgcactgtgtatttgctccgagcccagtcaaaagctcagggttcatctgttcccagggtgctgctattggtcagggggctcagagcccagatctgcagcacagcaggtcctgatactgctgagtccatgcagtgctgaaagggggaaaagggcatagagggaggagtctgctttgacagtgatgagaggtgggggtgaggctcctgacctccctgacccctttatccctgacagtccttctctggtccctctagctcagaaggctgggccccttgctgcttgaccttagagactgagagcgcaggaggcaaagccacaggtgtctttcagaaccagcctgagggagcaggctgtgaagacctgggagagaggtcttaaaataaatatcaatagtgatgtgaccctccccatatcttgcaaactgccagaccccctgagatactccctcctgttcctcagcccagttctgtcacaatgacgattttctgaaacctttatagggccacagaccactttatgaaactgttgtaagctatggatccaccccactcacaaaaatgtttgtgtgtacccacaaacacagtttagccagtttcaggggttcataaaccctaaagctcaggactctgatgagcctgatgagtacatagatcccatatcaatatcccattcttggctgggtgcggtggctcatgcctataatcacagcactttaggaggccgaggtgggaggagtgctcgaggccaggagttcaagaccagcctgggcaacatagtgagaccctgtctgtacaaaaataaaaaatttaaaaagtagctggctgtggtggtgcacacctgcagtcccagctacttgggaggccgaggtgggaggatcacttgagcctgggagattgaggctgcaataagctgtgtttgcaccactgcactgcagcctgggaaacagagcaagaccttgtctcaaaacaaacaaaaagaacccattctctcagcttctttgccttgtggacccaattttaccatgcagggctgtgatgaagcacacagagctttagcatctcaggtgggtcaggagagctacgatggccccagccctcagggcctgtgctgtcccagagggcagattcagagctgctgtggccttgagtttcactcttgaaccgaaggaatgctcaggggcaccactggccagcctgagacactggtctgataggaaagcaaatgagaagagaaggatggaaggaaggagcggaagggagtcggtgcattggaaatcacggagggctgccggtgcccttcacagaggctaggtttggtttctgtatctccctacatgtagcaggagtgcaaggtcactctgaccagctcagacatgcacttttggccaagcctctgggtaaaataatatacctccttttggcttagtgccttttgctggaaatctatcaatttcgggggactgtgctactggctaaccttgactccccactctttcccttttccctctttgtcctttggagacagtatttttctattctagcaaccacagccctcaatatggctggttcattctgtttcaggtgacttctgagggagcaaaggaaagaatgatgggaaaaaggtacaaggagacttcaggctctattgggctgaaggtcccagctccactgaacttaaatctaagtttacaacccagagagcagctcggtggtcagagcactgctgagccctactgcccctggtgggtcagggttgttgcaggactggctccctctcactgctctctggactcatcattttatctttcctaaaactcaggcttggtcaagtcactcatctccaataaaatctttgctgcctcccctttataaaatacagattcttcagtctggcattccagggacacctctgtagtctgacctgaacccacctgcccatgcctaccgttcgtaaatctcagtgtgcacccaatgatccagtcaccaagcctccctctgctccctgcacaggtaagctctccagctcagcacttcagtctgctacccttaggcctgggctattcttttttattttattttatttttatttttttgagacggagtctcattgtcgcccaggctggagtgcggtggcgctatctccgcccactgcaaccttcacctcctaggttcaagcaattctcctacctcagcctcccaagtagctgggattacaggcatgcgccaccacgcccgcctgatttttgtatttttagtagagacagggtttcccgtgttggccaggctggtctccaaccactgagctcaagtgaaccacccgcctcggcctcccaaagtgttgggattacaggcgtgagacatgtgcatctggttttccttattgaaaaatatttcccattcaatttaaaagctgtctccctgaggccttctccagcctttgtattctgaatttttctgcattcctcaatagtcattatccttggaatgaatacaattccaataacttttcactattttcagggctacctctcctagtagactgtaagctccttgagggcagacgccaggtttctggcactcagcccaatatctggcacagagtaggtgctcggtaaatgcctgtgaagtagcccatagactcccttacgcggtctgcaggacatgctgctctcctggcagcaccagcacagtctctaaatgctgccatatgcgagatatgtgtcaaccgctcaagcagccccggccttcttgagcgctccgcttctcagccaggtgcttattttgccagtgcccacgcccagtcaagaagaggacctaagggctcccctggatgtgtttgtttcaaacacaccttcagctttggagctgcagttttcttccgacctgctcggcaggcgggagggagctttcccctgaggctgggatcccatagggacccgcaccctgcaccctgcacccactggacgcaccagcctcataaaaaaactccccgcctccttcccccctccctttgtggacgcgcagcaattattctgcccattgccgtgaaaaagaagacaaaagttactttggcgccccctctcccacacctaatacagaggattcagggactctctggcgcttccagagcctgtgttagggacagaatccgcactggcgacggcgctccgactgcgcttctggcgacggtcggaattttgctcggccccttgcaatgtttccatgggaaggttcgtacattcgtgaccgtccctggcagcggcccagcccgggacttggcgcttcactcgccattggtcagtcctcggcgtgacgcgcaggggggcggggcctcatcagctgtttgcgggatgccccgagcaggcgta

SEQ ID NO: 16 - DNA, 4000 bp, Artificial Sequence
  chemically synthesized, IL1beta promoter sequence
gactgcttttctgaaatgatgcaaggttgagtagttgtacctgaaacatttattatctggccctttacagaaaatgtttccagaccctggataagtggtaccagagccccctctgtttgtggtcccctctcttatacccactaggtgtgagaaaagacatagagtaggagagccctgccatccatcttacccacccaggggctttttctgatggatccaaaggaaggacaaggtcttattggtctcccagaactgacataacaactccgacatcagggaaaagccattggagactacatagctcgccagccccagccacctgctcatatatctaagccctccttgttctagaccagggaggagaatggaatgtcccttggactctgcatgtccccaatctgagaacctggatccaagagggagaagaagcccattggagatgatgccataaaggaagtggaagcgatatgataaaaatcatagtgcccattcccaaataatcccagaagcagaagggaaaggagagaaatatccacaaagacaggtgtgggtacacacaacatttttcatactttaagatcccagagggactcatggaaatgatacaagaaaatgactcataagaacaaatattaggaagccagtgccaagaatgagatgggaaattggggaaaatgttgggggcagattgcttagttctgttctaagcaagagggtgaacaaggaaggaacagctcactacaaagaacagacatcactgcatgtacacacaataatataagaactaacccatgattattttgcttgtcttcttgttcaaaatgattgaagaccaatgagatgagatcaaccttgataactggctgcgaagcccatgattagacacaagatggtatcagggcacttgctgctttgaataaatgtcagtctcctgtcttggaagaatgacctgacagggtaaagaggaacttgcagctgagaaaggctttagtgactcaagagctgaataattccccaaaagctggagcatcctggcatttccagctccccatctctgcttgttccacttccttggggctacatcaccatctacatcatcatcactcttccactccctcccttagtgccaactatgtttatagcgagatattttctgctcattggggatcggaaggaagtgctgtggcctgagcggtctccttgggaagacaggatctgatacatacgttgcacaacctatttgacataagaggtttcacttcctgagatggatgggatggtagcagatttgggtccaggttacagggccaggatgagacatggcagaactgtggagactgttacgtcagggggcattgccccatggctccaaaatttccctcgagcgaaagcatcaggggctcatgcaacctggatactagtgctgcttcaaccacactgtgctattggatgagtcacttccaccctcctagccttgatttcttcgtctgctgttcacattcaaatagctattcatgtcttcatctctgtggtcccaccatatcccaccagacaatcattagggctcctcttagctggcagattctgaggtcctggatgctacaattggaagatggagaagtagaagctcaaggtttctgacctgtatcccaagtcccagaagcagaatggactaactcagagctgatgctcgggtcccttgcatatctcccttcctgtcactggctttgatcctccttcgttcagcttgtaatcacatcaacagaccaaagacatctctgtgttctgtcaggagagttcacagagccaccaaccctccagaccctgctggttgccgcataaagactctgaggaagggtttgaggctgctgtgatcatgcaatgaatgcatgattgtaccactgcactccagcctgggggataaaggtagatcctgtctaggagagagagagagagaaagagaaagagagagagaagggagggagagacaaagaaaaagagagagagggagggagaaagaaagagagaaagaaaagagaaaagaaagaaaaagaaagaaagagagagagggagggagggagagagaaagaaagaaagaaagagaaagagagaaagagagaaagagaaagaaaggaagaaagaaagaaagaaagaaagaaagaaagaaagaaagaaagaaaagaaaagaaagaaagagagagagaaagaaaaagaaagaggaaggaaggaaggaaggaagaaagacaggctctgaggaaggtggcagttcctacaacgggagaaccagtggttaatttgcaaagtggatcctgtggaggcaaaacagaggagtcccctaggccacccagacagggcttttagctatctgcaggaccagacaccaaatttcaggagggctcagtgttaggaatggattatggcttatcaaattcacaggaaactaacatgttgaacagcttttagatttcctgtggaaaatataacttactaaagatggagttcttgtgactgactcctgatatcaagatactgggagccaaattaaaaatcagaaggctgcttggagagcaagtccatgaaatgctctttttcccacagtagaacctatttccctcgtgtctcaaatacttgcacagaggctcactcccttggataatgcagagcgagcacgatacctggcacatactaatttgaataaaaatgctgtcaaattcccattcacccattcaagcagcaaactctaccacctgaatgtacatgccaggcactgtgctagacttggctcaaaaagatttcagtttcctggaggaaccaggaggagcaaggtttcaactcagtgctataagaagtgttacaggctggacacggtggctcacgcctgtaatcccaacactttgggaggccgaggcgggcagatcacaaggtcaggagatcgagaccatcctggctaacatggtgaaaccctgtctctactaaaaatacaaaaaattagccgggcgtggcggcaggtgcctgtagtcccagctgctggggaggctgaggcaggagaatggtgtgaacccgggaggcggaacttgcagggggccgagatcgtgccactgcactccagcctgggcgacagagtgagactctgtctcaaaaaaaaaaaaaaagtgttatgatgcagacctgtcaaagaggcaaaggagggtgttcctacactccaggcactgttcataacctggactctcattcattctacaaatggagggctcccctgggcagtaccctggagcaggcactttgctggtgtctcggttaaagagaaactgataactcttggttggtattaccaagagatagagtctcagatggatattcttacagaaacaatattccacttttcagagttcaccaaaaaatcattttaggcagagctcatctggcattgatctggttcatccatgagattggctagggtaacagcacctggtcttgcagggttgtgtgagcttatctccagggttgccccaactccgtcaggagcctgaaccctgcataccgtatgttctctgccccagccaagaaaggtcaattttctcctcagaggctcctgcaattgacagagagctcctgaggcagagaacagcacccaaggtagagacccacaccctcaatacagacagggagggctattggcccttcattgtacccatttatccatctgtaagtgggaagattcctaaacttaagtacaaagaagtgaatgaagaaaagtatgtgcatgtataaatctgtgtgtcttccactttgtcccacatatactaaatttaaacattcttctaacgtgggaaaatccagtattttaatgtggacatcaactgcacaacgattgtcaggaaaacaatgcatatttgcatggtgatacatttgcaaaatgtgtcatagtttgctactccttgcccttccatgaaccagagaattatctcagtttattagtcccctcccctaagaagcttcc

SEQ ID NO: 17 - DNA, 3001 bp, Artificial Sequence
  chemically synthesized, IGFBP3 promoter sequence
gtctattggtggtcagtaagactgatagccacaccccagaggacactgagaaacagtagtgctgtcagaaggagagagaccctagggtggcttgtccagctgggggctgagcagaagaaaagcaaaggagccagtatcacgcagcgacaaggtgccctttagcagcaggagtgggctcaaagaaatgagtggtttaggtcacctggtgaaaataacgaggctgaccaaaggaaacactaattataatcaggtcagctggactccgttagtgaaaagagatctatgcagcctcctgacctctattcggatggttattcgctacttttgcaatgattagcaattgtatgccaagcaccttcacagggggtaaacatctttctcactaaatactagtttatgttaaaaactacaaacaccagatgaattggaagtttaacagaattaacgagcatgtcacaaaattaagttttatccagtccaaatccttcctgcaacaattatgttccgggaggggcagggagcttgcagttatcaaataaaactggctaaataacaaaacacaattctccccgtgtcactgcctcaaatttactctgctgttgcaagatgtcatgataaaggtgatggcaatctatataaaacattgttattggaatagcctttgaaagttgaaatgtgacttagtctttacgtgtgtcccgtggtccctgaggccgggctggacgcctggagctttggcgccccctagccattcggcactgaacaagtttctccaactgcaatgcgagggaaaaaagaagagaaccaagaataaaacaaattttttaatcctttccaaaaagacattgtttctgatcttcccctgtccactctccagatgataaagctgaataaaaagtgctacactaaccagtggtcagtccaggctctgctgcaacattgcactaaccagaactgcagacctgggacctcaagaattgcatttgatgccgaacccagctctaatttcagagtcaaggtctctgcgagtatttaaggaacggatgtaaacctgggggattcgttttgtttccttcaattttccaatgaaatcagagatcctgttcttgggtgtcaacgcagatactagaaggaggtgatacaagagaaaggaaacagcaagcgacgattatggcacggtttcctgtaaacaaggttgagtgtagccacagcctgagcactgtgggagaagagctcataagaaaatgacggtgctgggccttcgtcaccccggggcactccattgttcttgtctttggtctctttttatttgtagaggtccaattatttatttatttagtacaagagggaacgaaattgatctttccattctaaaaggagagtatatatgtataaaaggaagctgtatagatatgggggaagaggtggacagggggaaaaggggagaggacgagagagagaaagggagggagagggacaaggagagacactgggcgagagatcgattaggagagacagaaatgatgaatgaagattaacttcacccaaggcttcgtgctggaggggaatggaggagctcctgatttgctattactactccaaactgcaaagggctccttcaagtcacctatccacctcctaaggcaagcgtccaatttcaacagcgttcaggaaagtctcctcccgcggaggtctcaccgcttcccactccacccccacaaactctttggaaaagtgccttgaaaaatttaatcctcaatccaatcctggaccaccagcgtcctctgttggtcaccgaagggagggggtgcgcagacaaaactgaagaaactcgagtgccagagaaggccgacaggagttacagcgacctcagcgcgcaattgcgcccccgaactttactgaaaagtgtttagattgcagagataagctagaatcccaacgcatcgagaatacagtaatacgaagtcgccttcaaaaaatgacaatgaaaattgcctattaaaggactatttggttaattacgtttcagcagtgcccagtttattgtctttattattcttttgtcatgggtgtaaactccatttgaaaacataatcagggagaatacccaagacaagaagaacagttgtcatttaaaatatttgaaaagccctgccttaaggacgcattcgcttgccggtccactcttaattggagacttgcggtgtagcaacacgtgagagtcttcttgcgttgagaagtaagcctggaaaggggcgaaggccccgggcgcatcttcagatgcgtatttgtgggcccctggggatataaacagcccagcgggtgtaaattaaaccccgcagtgccttggctccctgagacccaaatgtaagtcagaaatgtcccaagacttcgcctgccaacggaattaaattttagaaagctccacgaggtacacacgaatgcggagcgctgtatgccagtttccccgacaccggctcgccgcagggagacctcaccccgagagcggaaggggtaagggcggcggggtcaaggagatcgggggtgctgagttggccaggagtgactggggtgaccgggggtgctgaggtggcctggagtgccggggtggccgggcacaccttggttcttgtagacgacaaggtgacccgggctccgggcgtgcgcacgaggagcaggtgcccgggcgagtctcgagctgcacgcccccgagctcggccccggctgctcagggcgaagcacgggcccccgcagccgtgcctgcgccgacccgcccccctcccaacccccactcctgggcgcgccgttccggggcgtgtcctgggccaccccggcttctatatagcggccggcgcgcccgggccgcccagatgcgagcactgcggctgggcgctgaggatcagccgcttcctgcctggattccacagcttcgcgccgtgtactgtcgccccatccctgcgcgcccagcctgccaagcagcgtgccccggttgcaggcgtca

SEQ ID NO: 18 - DNA, 46 bp, Artificial Sequence
  chemically synthesized, BMF cloning primer
gcggcggatatctaataagtgtttgcaccactgcactgcagcctgg

SEQ ID NO: 19 - DNA, 41 bp, Artificial Sequence
  chemically synthesized, Luciferase cloning primer
gcggcgtctagattacacggcgatcttgccgcccttcttgg

(19) United States
(12) Patent Application Publication
(10) Pub. No.: US 2020/0109434 A1
(43) Pub. Date: Apr. 9, 2020

(54) Compositions and Methods for Diagnosing Recurrent Focal and Segmental Glomerulosclerosis (rFSGS)

(71) Applicant: MUSC Foundation for Research Development, Charleston, SC (US)

(72) Inventors: Deepak Nihalani, Mount Pleasant, SC (US); Pankaj Srivastava, Charleston, SC (US); Milos Budisavljevic, Charleston, SC (US); Michael G. Janech, Charleston, SC (US); Peifeng Deng, Charleston, SC (US); Ashish K. Solanki, Charleston, SC (US); Ehtesham Arif, Charleston, SC (US)

(21) Appl. No.: 16/597,276

(22) Filed: Oct. 9, 2019

Related U.S. Application Data
(60) Provisional application No. 62/743,447, filed on Oct. 9, 2018.

(51) Int. Cl. C12Q 1/66; C12Q 1/6876; C12N 15/85

(52) U.S. Cl. CPC C12Q 1/66; C12N 15/85; C12Q 1/6876

(57) ABSTRACT

The present invention relates to disease-responsive reporter constructs and their use for diagnosing diseases, including recurrent focal segmental glomerulosclerosis (rFSGS).

### CROSS REFERENCE TO RELATED APPLICATIONS

This application claims priority to U.S. Provisional Application No. 62/743,447, filed Oct. 9, 2018 which is hereby incorporated by reference herein in its entirety.

### BACKGROUND OF THE INVENTION

Focal and segmental glomerulosclerosis (FSGS) is a glomerular disease that primarily targets kidney podocytes which are the important constituents of kidney's filtration barrier and whose dysfunction leads to proteinuria and progressive renal failure.

Although FSGS is one of the leading causes of end-stage renal disease (ESRD), or kidney failure, advancements in diagnosing and therapeutic treatment of this disease remain inadequate. Although tissue biopsies are expensive and involve invasive procedures, they are a gold standard of diagnosis for many renal diseases. For glomerular diseases such as FSGS, no other diagnostic procedure closely matches the specificity of a renal biopsy. Recurrent FSGS (rFSGS) patients, who continue to suffer from the lack of transplant success, are relinquished to dialysis. There are no reliable methods upon which to predict primary FSGS, and unsuccessful kidney transplants due to primary FSGS are resource heavy and with lesser benefit. Thus an assay that would identify rFSGS condition would be immensely beneficial in not only reducing the overall cost of unsuccessful renal transplants, but also providing insight into the mechanism of this disease that will advance treatment options for these patients. It is estimated that approximately 1,000 FSGS patients receive a kidney transplant each year; however, more than one third of these FSGS patients suffer from recurrence of the disease within hours to weeks of transplant. This not only produces an emotional but also financial burden on patients and their insurance companies including Medicare.

In recent years, substantial insight in the pathogenesis and genetics of FSGS have accumulated but not in the diagnosis. Significant effort has been spent on analyzing FSGS patient sera/plasma, urine or biopsies to develop the diagnostic tool of FSGS but the gold standard remains the highly invasive and cumbersome method of kidney biopsy (Sprangers et al., 2016, Biomed Res Int. 2016:4632768). Further, it is not uncommon that after an initial biopsy showing no clear FSGS lesion a subsequent biopsy taken months or years later shows clear FSGS lesions (Howie et al., 2005, Kidney International, 67(3):987-1001). ELISA based detection of pathogenic antibodies for rFSGS diagnosis has been reported but this assay is not very robust, is very expensive and therefore cannot be used in a clinical setting (Delville et al., 2014, Sci Transl Med. 6(256):256ra136). Since the existing diagnosis of rFSGS requires an invasive procedure known as renal biopsy, there is an urgent need to develop an alternate assay that can be performed either at the hospital or at any tertiary care centers.

Predicting recurrence of FSGS has been a challenging quest for many investigators due to unknown features of humoral factor(s) that are likely responsible for FSGS recurrence and loss of renal allograft (Savin V. J. et al., 2012, Kidney Research and Clinical Practice, 31(4):205-213). Several approaches have been developed but none of them could be successfully adopted as a routine diagnostic tool in detecting rFSGS. Although the discovery of suPAR as a circulating factor and a biomarker for rFSGS gained a lot of attention, subsequent studies (Wei, C. et al., 2011, Nat Med., 17(8):952-960; Gallin, L. et al., 2017, Nat Rev Nephrol., 13(9):593; Kronbichler, A. et al., 2016, J Immunol Res., p. 2068691) concluded that suPAR could be the marker of progression of nephrotic syndrome, but not specifically the FSGS or rFSGS (Kronbichler, A. et al., 2016, J Immunol Res., p. 2068691; Spinale, J. M. et al., 2015, Kidney Int., 87(3):564-574), and hence its use in clinical diagnosis of rFSGS remains questionable. Apart from suPAR, glomerular enlargement assay (Lee, H. S. et al., 1995, Clin Nephrol., 44(6):349-355), an antibody (Delville, M. et al., 2014, Sci Transl Med., 6(256):p. 256ra136) and cell-culture based approaches were proposed to diagnose rFSGS. However, these assays are time consuming, technically challenging, and involve a panel of antibodies that make them expensive to perform and therefore, unlikely to be adopted commercially. Moreover, due to these limitations these assays will not likely be tested against large sample databases.

Thus, there remains a need in the art for assays for diagnosing rFSGS. The present invention fulfills these needs.

### SUMMARY OF THE INVENTION

In one embodiment, the invention relates to a composition comprising a disease-responsive reporter construct. In one embodiment, the construct comprises at lease one promoter of a disease-responsive gene operably linked to at least one sequence encoding a reporter molecule.

In one embodiment, the promoter of a disease-responsive gene comprises at least 250 nucleotides immediately upstream of a disease-responsive gene start codon.

In one embodiment, the disease is the recurrence of a nephrotic syndrome (RNS), and the RNS-responsive gene is ABCC6, AC005682.5, AC084018.1, ADAMTS1, ADAMTS5, ASB2, BAAT, BMF, CCDC147, CCL2, CDKN1C, CDKN2B, CHAC1, CSAD, CSGALNACT1, CYP1B1, CYP24A1, DDIT3, DTX3, FBXLS, GREM1, GSDMB, HCPS, HLA-DMB, HSD17B7P2, IGFBP3, IGFBP5, IL-11, IL-1A, IL-1β, KCTD19, KCTD4, KRT23, KRTAP1-1, KRTAP2-3, LINC00263, MAP3K7CL, NEAT1, NPPB, NR1D2, PLA2G4C, PLK1S1, PTGIS, RAPGEF3, RNF207, RP11-16112.1, RP11-336N8.4, RP5-1021120.2, RPL13AP20, RRNAD1, RUSC1-AS1, SEMA3A, SLC22A3, SLC25A27, SLC7A14, SMOC1, SMPD3, SUSD4, TMEM86A, TNF, TNFSF10, TRIM45, TRPC4, WDR66, XXbac-BPG246D15.8, TMEM2, KIAA0247, RERE, PDXK, TAP1, CARHSP1, NEDD9, TAPSAR1, CPD, CNDP2 or a combination thereof.

In one embodiment, the RNS-responsive gene is IGFBP3, IL-1β or BMF. In one embodiment, the promoter has a nucleotide sequence as set forth in SEQ ID NO:15, SEQ ID NO:16 or SEQ ID NO:17, or a variant or fragment thereof. In one embodiment, the nucleotide sequence as set forth in SEQ ID NO:15, SEQ ID NO:16 or SEQ ID NO:17, or a variant or fragment thereof, is operably linked to luciferase. In one embodiment, the nucleotide sequence as set forth in SEQ ID NO:15, SEQ ID NO:16 or SEQ ID NO:17, or a variant or fragment thereof, is on a plasmid. In one embodiment, the nucleotide sequence as set forth in SEQ ID NO:15, SEQ ID NO:16 or SEQ ID NO:17, or a variant or fragment thereof, is integrated into the genome of a cell.

In one embodiment, the invention relates to a cell comprising a disease-responsive reporter construct. In one embodiment, the construct comprises a promoter of a disease-responsive gene operably linked to at least one sequence encoding a reporter molecule. In one embodiment, the reporter molecule is luciferase. In one embodiment, the reporter construct is a plasmid. In one embodiment, the reporter construct is integrated into the genome of the cell.

In one embodiment, the invention relates to a method of diagnosing a disease in a subject, the method comprising: obtaining a test sample of the subject, contacting a cell comprising at least one disease-responsive reporter construct with the test sample, measuring the expression level of at least one reporter molecule, comparing the expression level of at least one reporter molecule to the level of a comparator control, and diagnosing the subject as having or at risk of the disease or disorder to which the disease-responsive reporter construct is responsive when the level of expression of at least one reporter construct is altered as compared to a comparator control. In one embodiment, the disease-responsive reporter construct comprises a promoter of a disease-responsive gene operably linked to a sequence encoding at least one reporter molecule. In one embodiment, the promoter of a disease-responsive gene comprises at least 250 nucleotides immediately upstream of a disease-responsive gene start codon.

In one embodiment, method is a method of diagnosing recurrence of a nephrotic syndrome (RNS), and the RNS-responsive gene is ABCC6, AC005682.5, AC084018.1, ADAMTS1, ADAMTS5, ASB2, BAAT, BMF, CCDC147, CCL2, CDKN1C, CDKN2B, CHAC1, CSAD, CSGALNACT1, CYP1B1, CYP24A1, DDIT3, DTX3, FBXLS, GREM1, GSDMB, HCPS, HLA-DMB, HSD17B7P2, IGFBP3, IGFBP5, IL-11, IL-1A, IL-1β, KCTD19, KCTD4, KRT23, KRTAP1-1, KRTAP2-3, LINC00263, MAP3K7CL, NEAT1, NPPB, NR1D2, PLA2G4C, PLK1S1, PTGIS, RAPGEF3, RNF207, RP11-16112.1, RP11-336N8.4, RP5-1021120.2, RPL13AP20, RRNAD1, RUSC1-AS1, SEMA3A, SLC22A3, SLC25A27, SLC7A14, SMOC1, SMPD3, SUSD4, TMEM86A, TNF, TNFSF10, TRIM45, TRPC4, WDR66, XXbac-BPG246D15.8, TMEM2, KIAA0247, RERE, PDXK, TAP1, CARHSP1, NEDD9, TAPSAR1, CPD, CNDP2 or a combination thereof. In one embodiment, the RNS-responsive gene is IGFBP3, IL-1β or BMF. In one embodiment, the promoter has a nucleotide sequence as set forth in SEQ ID NO:15, SEQ ID NO:16 or SEQ ID NO:17. In one embodiment, the nucleotide sequence as set forth in SEQ ID NO:15, SEQ ID NO:16 or SEQ ID NO:17, or a variant or fragment thereof, is operably linked to luciferase. In one embodiment, the nucleotide sequence as set forth in SEQ ID NO:15, SEQ ID NO:16 or SEQ ID NO:17, or a variant or fragment thereof, is on a plasmid. In one embodiment, the nucleotide sequence as set forth in SEQ ID NO:15, SEQ ID NO:16 or SEQ ID NO:17, or a variant or fragment thereof, is integrated into the genome of a cell.

In one embodiment, the comparator control is at least one of: a positive control, a negative control, a historical control, a historical norm, or the level of a reference molecule in the biological sample.

In one embodiment, the subject is human.

In one embodiment, the RNS is recurrent focal and segmental glomerulosclerosis (rFSGS). In one embodiment, the method further comprises the step of administering a treatment for rFSGS to the subject.

In one embodiment, the method comprises differentially diagnosing a subject as having rFSGS.

In one embodiment, the invention relates to a method of identifying a disease-responsive reporter gene, comprising: contacting cells with a biological sample from a subject having a disease or disorder for which a disease-responsive reporter construct is desired; isolating RNA from the cells; and performing an analysis on the isolated RNA to identify candidate genes whose expression level was altered in the presence of the biological sample from the subject having the disease or disorder.

In one embodiment, the invention relates to a method of constructing a disease-responsive reporter comprising: identifying the promoter region of one or more disease-responsive reporter gene; and generating a disease-specific reporter construct, wherein the reporter construct comprises a nucleotide sequence comprising the promoter of the candidate gene operably linked to a nucleotide sequence encoding one or more reporter markers.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 depicts a flow diagram of the experimental design used to identify rFSGS responsive candidate gene promoters and develop rFSGS reporter constructs and cell lines. Total RNA was isolated from podocytes treated with rFSGS and control patient plasma and subjected to mRNA profiling. Candidate upregulated genes with proapoptotic function were selected from the list of DEGs (p_{adj}<0.05). Promoter regions from the candidate genes were cloned in a luciferase reporter vector and stable cell lines were constructed using puromycin as a selection marker. Stable cell lines expressing the promoter-driven reporter were treated with plasma from control or rFSGS patients and the reporter activity was measured using ONE-Glo™ EX Luciferase Assay System (Promega). Fold changes were calculated after normalizing with the control plasma.

FIG. 2 depicts a Venn diagram showing 483 common upregulated genes were obtained from RNA Seq data analysis.

FIG. 3 depicts a list of select top upregulated genes identified from an analysis of RNA Seq data.

FIG. 4A and FIG. 4B depict the results of exemplary experiments demonstrating PCR amplification of the promoter regions that were used to develop rFSGS reporter constructs. FIG. 4A depicts the results of exemplary experiments demonstrating the PCR amplification of the BMF promoter. FIG. 4B depicts the results of exemplary experiments demonstrating the PCR amplification of the IL-1β promoter.

FIG. 5 depicts a diagram of the pGL4.20[luc2/Puro] promoterless vector.

FIG. 6 depicts a flow diagram demonstrating a method of using the rFSGS reporter construct in an assay for identifying subjects having rFSGS.

FIG. 7A and FIG. 7B depict the results of exemplary experiments demonstrating that the plasma from rFSGS patients induces podocyte actin cytoskeleton damage. FIG. 7A depicts actin cytoskeleton staining. Plasma from rFSGS patients induced actin cytoskeletal reorganization. Representative images of the actin cytoskeleton staining with phalloidin and DAPI of the podocytes treated with rFSGS patient or control plasma are shown in the left panel, FSGS and MGN patients are shown in the right panel. FIG. 7B depicts quantification of altered actin cytoskeleton morphology depicting cellular damage upon treatment of podocytes with rFSGS patients or control plasma. The cellular damage was assessed at >80% in podocytes treated with rFSGS patient plasma, whereas for FSGS and MGN plasma treated podocytes, <30% overall cellular damage was observed (p≤0.05, 2-tailed t-test, >10 cells per experimental condition were evaluated from three experimental repeats). Bar=25 *P<0.05, **P<0.01 and ***P<0.01 according to the unpaired t test.

FIG. 8 depicts quantitative RT PCR of upregulated candidate genes. qPCR analysis using BMF, IL1β and IGFBP3 gene specific primers showed upregulation of candidate genes in rFSGS plasma (from patients rF A-J) treated podocytes, whereas minimal or no upregulation was noted in FSGS (Fs A-E) treated podocytes. The unpaired t test was performed (*P<0.05, **P<0.01 and ***P<0.01).

FIG. 9A through FIG. 9E depict the results of exemplary experiments demonstrating that the cell lines containing firefly receptor constructs selectively responded to rFSGS patient plasma. FIG. 9A depicts exemplary experimental results demonstrating that the constructed BMF reporter-based podocyte cell line selectively responded to rFSGS patient plasma but not to control plasma or plasma derived from other nephropathy patients. FIG. 9B depicts exemplary experimental results demonstrating that the constructed IL1β reporter-based podocyte cell line selectively responded to rFSGS patient plasma but not to control plasma or plasma derived from other nephropathy patients. FIG. 9C depicts exemplary experimental results demonstrating that the constructed IGFBP3 reporter-based podocyte cell line selectively responded to rFSGS patient plasma but not to control plasma or plasma derived from other nephropathy patients. Treatment of reporter cell lines BMF, IL1β and IGFBP3 with plasma from majority of rFSGS patients (rF A-N) showed elevated reporter activity (˜1.5-2.5 fold increase over control), whereas the MGN plasma (MGN A, B) and MG (MG1) plasma showed no response. FIG. 9D depicts exemplary experimental results demonstrating that the constructed negative control, LAMP3 gene promoter reporter cell line did not respond to plasma from control patient, rFSGS (rF A, D, E), FSGS (Fs A-E) and MGN (MGN A, B). CD63 Ab was used as a positive control (PC), to induce Lamp3 promoter reporter activity and untreated control cells were used as negative control. FIG. 9E depicts exemplary experimental results demonstrating the specificity of the reporter constructs when treated with plasma from non-rFSGS (Fs A-P) patients. The maximal response was noted only from the rFSGS patient A (rF A), which was used as a positive control. The results are expressed as fold change in light units over control plasma, which is the ratio of absolute light units of the experimental samples to that of control plasma treated cell lines. Results represent 3 independent biological repeats and 2 technical repeats.

FIG. 10A through FIG. 10D depict the results of exemplary experiments demonstrating plasma induced reporter activity is independent of host cell line. FIG. 10A depicts exemplary experimental results from the transfection of the promoter reporter constructs into HEK293 cells. FIG. 10B depicts exemplary experimental results from the transfection of the promoter reporter constructs into Cos7 cells. Similar to the chimeric podocyte cell lines, these cell lines also responded selectively to the plasma from rFSGS patients (A to E) but not to the control, FSGS (A to E) or MGN/MG patient/s plasma. FIG. 10C and FIG. 10D depict the mean fold-change response from BMF and IL1β promoter HEK and Cos7 cell lines. The data were categorized into three groups (FSGS; rFSGS and MGN/MG plasma) and the statistics were done using one way ANNOVA with multiple comparison (*P<0.05, **P<0.01 and ***P<0.0). The p values within the group have been calculated according to the unpaired t test.

FIG. 11A through FIG. 11D depict the results of exemplary experiments demonstrating that the cell-based assay detects rFSGS patents with high sensitivity and specificity. FIG. 11A depicts exemplary experimental data demonstrating the differences in mean fold-change response from BMF, IL1β and IGFBP3 cell lines between rFSGS patient samples and other nephropathies. The data was statistically analyzed after pooling and was categorized into three groups (FSGS; rFSGS and MGN/MG/Control plasma). The fold change for BMF, IL1β and IGFBP3 promoter cell lines treated with rFSGS plasma were significantly higher when compared to non-rFSGS/MGN patient plasma. Points are values averaged within subject for each group and the lines represent the 95% confidence interval for mean FC. The horizontal dashed line represents 1-fold change (control). P-values beside the points for each group represent the p-value for comparison to control. Significant differences for pairwise group comparisons are signified by the vertical lines where * is p<0.05, ** is p<0.01, and *** is p<0.001. All p-values are corrected for multiple comparisons using Tukey's honestly significant difference. In contrast, no significant change in the expression of LAMP3 relative in rFSGS samples vs other kidney diseases were observed (p=0.999). Additionally, no differences were observed between samples from patients with FSGS or other nephropathies in this cell line. FIG. 11B through FIG. 11D depict exemplary analyses demonstrating the discriminative performance for each promoter. Sensitivity and specificity values for IL1β, BMF, and IGFBP3 promoter cell lines were estimated using receiver operator characteristic curves. The AUCs for model fits discriminating between rFSGS and all other nephropathies and between rFSGS and non-recurrent FSGS ranged from 0.81 to 0.86 respectively, for IL1β and BMF reporter cell lines, whereas these were slightly lower for the IGFBP3 cell line (70% and 64% respectively). AUC, area under the curve.

FIG. 12 depicts demographic and treatment characteristics of all patients.

FIG. 13 provides a table demonstrating that the reporter constructs can be used to discriminate between disease conditions. 95% confidence intervals are shown for differentiating between rFSGS vs. all other nephropathies and rFSGS vs. non-recurrent FSGS.

FIG. 14 depicts a schematic diagram of the Crispr/Cas9 system for generating stable cell lines in which the reporter construct is integrated into the genome.

FIG. 15 depicts exemplary experimental data demonstrating that a Crspr/Cas9 constructed HEK reporter-based cell line selectively responded to rFSGS patient plasma (rFA-E) but not to control plasma or plasma derived from other nephropathy patients.

FIG. 16 depicts exemplary experimental data demonstrating that the BMF reporter cell line was incubated with patient plasma for indicated times and the assay response was measured.

FIG. 17 depicts exemplary experimental data demonstrating the construction of CRSPR/Cas-based BMF promoter-reporter cell line in HEK293 cells.

FIG. 18 depicts exemplary experimental data demonstrating that the BMF CRSPR/Cas9 reporter cell line was incubated with patient plasma for 4 hours and the assay response was measured.

### DETAILED DESCRIPTION OF THE INVENTION

In one embodiment, the invention provides methods for developing disease-responsive reporter constructs for use in diagnosing a desired disease. In one embodiment, the method includes: a) contacting cells with a biological sample from a subject having a disease or disorder for which a disease-responsive reporter construct is desired, b) isolating RNA from cells derived from the biological sample, c) performing an analysis on the isolated RNA to identify candidate genes whose expression level was altered in the presence of the biological sample from the subject having the disease or disorder, d) identifying the promoter region of one or more candidate genes, and e) constructing a disease-specific reporter construct comprising the promoter of the candidate gene operably linked to a nucleotide sequence encoding one or more reporter markers.

In one embodiment, the invention relates to disease-responsive reporter constructs for use in methods of diagnosing a disease or disorder in a biological sample of a subject. In another embodiment, the invention relates to vectors and cells comprising a disease-responsive reporter constructs for use in methods of diagnosing a disease or disorder in a biological sample of a subject. In one embodiment, a disease-response promoter of the invention provides a nucleic acid molecule comprising at least one promoter sequence of a protein that is altered (i.e., upregulated or downregulated) upon contact with a biological sample from a subject with a disease to be detected or diagnosed, operably linked to at least one reporter marker.

In one embodiment, the present invention relates to the discovery that plasma from patients with a disease or disorder contains signals that can promote expression of particular genes associated with the disease or disorder. Thus, the present invention relates to compositions and methods useful for the assessment, diagnosis, characterization, prevention and treatment of a disease or disorder.

In one embodiment, the compositions of the invention relate to disease-responsive reporter constructs comprising a promoter region of a gene that has altered expression upon contact with a biological sample from a subject who has the disease or disorder operably linked to a reporter molecule.

In one embodiment, the disease or disorder is a recurrent nephrotic syndrome (RNS), including, but not limited to, glomerular diseases, rFSGS and podocyte malfunction, and the invention provides RNS-responsive reporter constructs. In one embodiment, a gene that has increased expression in response to contact with a sample from a subject with a RNS is one of IGFBP3, IL-1β and BMF. Therefore, in one embodiment, the recurrent nephrotic syndrome-responsive reporter constructs of the invention comprise a promoter of at least one of IGFBP3, IL-1β and BMF operably linked to at least one reporter molecule.

In one embodiment, the invention relates to cell lines comprising the disease-responsive reporter constructs of the invention, and methods of using the cell lines for the diagnosis of a disease or disorder. In one embodiment, the disease-responsive reporter constructs of the invention are on a plasmid. In one embodiment, the disease-responsive reporter constructs of the invention are integrated into the genome of the cell. In one embodiment, the disease or disorder is a RNS, and the invention provides cells comprising at least one RNS-responsive reporter construct. In one embodiment, a gene that has increased expression in response to contact with a sample from a subject with RNS is one of IGFBP3, IL-1β and BMF. Therefore, in one embodiment, the cells comprise one or more RNS-responsive reporter constructs of the invention comprising a promoter of at least one of IGFBP3, IL-1β and BMF operably linked to at least one reporter molecule.

In one embodiment, the invention provides methods of diagnosing a disease or disorder using the reporter constructs and cells of the invention. In one embodiment, the methods include the steps of contacting a cell comprising a disease-responsive reporter construct with a biological sample from a subject, measuring expression of the reporter molecule of the disease-responsive reporter construct and diagnosing the subject as having the disease or disorder for which the reporter construct is responsive to when the expression of the reporter molecule of the disease-responsive reporter construct is altered as compared to a comparator control.

In one embodiment, the compositions and methods of the invention are useful for diagnosing a subject as having or at risk of a RNS, including, but not limited to, glomerular diseases, rFSGS and podocyte malfunction. In one embodiment, the compositions and methods of the invention are useful for diagnosing a subject as having or at risk of recurrence of a nephrotic syndrome after a transplant.

In one embodiment, the invention provides methods of diagnosing recurrence of a nephrotic syndrome in a subject in need thereof comprising contacting at least one cell comprising at least one RNS-responsive reporter construct with a sample of a subject, measuring expression of the reporter molecule of the disease-responsive reporter construct, and diagnosing the subject as having or at risk of recurrence of the nephrotic syndrome when the expression of the construct is altered as compared to a comparator control. In one embodiment, a gene that has increased expression in response to contact with a sample from a subject with recurrence of a nephrotic syndrome is one of IGFBP3, IL-1β and BMF. In one embodiment, the RNS is rFSGS.

In one embodiment, the compositions and methods of the invention are useful for diagnosing a subject as having or at risk of RNS. In one embodiment, the compositions and methods of the invention are useful for differentially diagnosing a subject as having RNS, wherein the differential diagnosis is made between RNS (e.g., rFSGS) and a non-recurrent nephrotic syndrome (e.g., FSGS). In one embodiment, the compositions and methods of the invention are useful for differentially diagnosing a subject as having RNS, wherein the differential diagnosis is made between RNS and another nephropathy.

### Definitions

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

An “allele” refers to one specific form of a genetic sequence (such as a gene) within a cell, an individual or within a population, the specific form differing from other forms of the same gene in the sequence of at least one, and frequently more than one, variant sites within the sequence of the gene. The sequences at these variant sites that differ between different alleles are termed “variants.” “polymorphisms,” or “mutations.”

As used herein, to “alleviate” a disease or disorder, such as RNS, means reducing the frequency or severity of at least one sign or symptom of a disease or disorder.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

The term “derived from” is used herein to mean to originate from a specified source.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

A “fluorophore” is a component of a molecule which causes a molecule to be fluorescent. It is a functional group in a molecule which will absorb energy of a specific wavelength and re-emit energy at a specific wavelength. The amount and wavelength of the emitted energy depend on both the fluorophore and the chemical environment of the fluorophore. Fluorescein isothiocyanate (FITC), a reactive derivative of fluorescein, has been one of the most common fluorophores chemically attached to other, non-fluorescent molecules to create new fluorescent molecules for a variety of applications. Other historically common fluorophores are derivatives of rhodamine (TRITC), coumarin, and cyanine. Newer generations of fluorophores such as the CF dyes, the FluoProbes dyes, the DyLight Fluors, the Oyester dyes, the Atto dyes, the HiLyte Fluors, and the Alexa Fluors are also known in the art.

The term “gene” refers to a nucleic acid (e.g., DNA) sequence that includes coding sequences necessary for the production of a polypeptide, precursor, or RNA (e.g., mRNA). The polypeptide may be encoded by a full length coding sequence or by any portion of the coding sequence so long as the desired activity or functional property (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 4 kb or more on either end such that the gene corresponds to the length of the full-length mRNA and 5′ regulatory sequences which influence the transcriptional properties of the gene. Sequences located 5′ of the coding region and present on the mRNA are referred to as 5′-untranslated sequences. The 5′-untranslated sequences usually contain the regulatory sequences. Sequences located 3′ or downstream of the coding region and present on the mRNA are referred to as 3′-untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

A “genome” is all the genetic material of an organism. In some instances, the term genome may refer to the chromosomal DNA. Genome may be multichromosomal such that the DNA is cellularly distributed among a plurality of individual chromosomes. For example, in human there are 22 pairs of chromosomes plus a gender associated XX or XY pair. DNA derived from the genetic material in the chromosomes of a particular organism is genomic DNA. The term genome may also refer to genetic materials from organisms that do not have chromosomal structure. In addition, the term genome may refer to mitochondria DNA. A genomic library is a collection of DNA fragments representing the whole or a portion of a genome. Frequently, a genomic library is a collection of clones made from a set of randomly generated, sometimes overlapping DNA fragments representing the entire genome or a portion of the genome of an organism.

As used herein, the term “growth medium” is meant to refer to a culture medium that promotes growth of cells. A growth medium will often contain animal serum. In some instances, the growth medium may not contain animal serum.

“Identical” or “identity” as used herein in the context of two or more nucleic acid or polypeptide sequences means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

As used herein, an “immunoassay” refers to any binding assay that uses an antibody capable of binding specifically to a target molecule to detect and quantify the target molecule.

“Instructional material,” as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains the nucleic acid, peptide, and/or compound of the invention or be shipped together with a container that contains the nucleic acid, peptide, and/or compound. Alternatively, the instructional material may be shipped separately from the container with the intention that the recipient uses the instructional material and the compound cooperatively.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated cell” refers to a cell which has been separated from other components and/or cells which naturally accompany the isolated cell in a tissue or mammal.

The term “modulate” or “modulation”, when used in reference to a functional property or biological activity or process (e.g., enzyme activity or receptor binding), refers to the capacity to either up regulate (e.g., activate or stimulate), down regulate (e.g., inhibit or suppress) or otherwise change a quality of such property, activity or process.

A “nucleic acid” refers to a polynucleotide and includes poly-ribonucleotides and poly-deoxyribonucleotides. Nucleic acids according to the present invention may include any polymer or oligomer of pyrimidine and purine bases, e.g., cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, at least 8, at least 15 or at least 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this disclosure. It will be understood that when a nucleotide sequence is represented herein by a DNA sequence (e.g., A, T, G, and C), this also includes the corresponding RNA sequence (e.g., A, U, G, C) in which “U” replaces “T”. As used herein, includes cDNA, RNA, DNA/RNA hybrid, antisense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

The term “reporter gene” or “reporter” is known in the art and as used in the present invention with respect to a DNA sequence means any DNA sequence encoding a peptide, a protein or a polypeptide or nucleic acid that can give rise to a signal that can be detected, traced, or measured. As used in the present invention with respect to a DNA sequence, “reporter” will generally means a cDNA sequence (although in some cases a reporter gene may have introns) that encodes a protein or polypeptide or nucleic acid that is used in the art to provide a measurable phenotype that can be distinguished over background signals. The product of said reporter gene may also be referred to a “reporter” and may be mRNA, a peptide, a polypetide, or protein, and may also be readily measured by any mRNA or protein quantification technique known in the art. “Reporter” may also refer to a tag or label that is affixed to a protein or peptide after it is expressed and may be any such tag or label known in the art. The reporter may, in a preferred embodiment, be a fluorophore.

The terms “patient,” “subject,” “individual,” and the like are used interchangeably herein, and refer to any animal, or cells thereof whether in vitro or in situ, amenable to the methods described herein. In certain embodiments, the patient, subject or individual is a human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Sample” or “biological sample” as used herein means a biological material isolated from a subject. The biological sample may contain any biological material suitable for detecting a physiologic or pathologic process in a subject, and may comprise fluid, tissue, cellular and/or non-cellular material obtained from the individual. One example of a biological sample is a plasma sample.

A sample may be characterized as “positive” for a particular disease or disorder. A positive sample is one wherein the sample is derived from a subject that is characterized as having or likely having the disease or disorder.

A sample may be characterized as “negative” for a particular disease or disorder. A negative sample is one wherein the sample is derived from a subject that is characterized as not having or likely not having the particular disease or disorder.

As used herein, the terms “therapy” or “therapeutic regimen” refer to those activities taken to alleviate or alter a disorder or disease, such as a RNS, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

To “treat” a disease as the term is used herein, means to reduce the frequency or severity of at least one sign or symptom of a disease or disorder, such as a RNS, experienced by a subject.

A “variant” may be a nucleic acid sequence that is substantially identical over the full length of the full gene sequence or a fragment thereof. The nucleic acid sequence may be 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The invention is based, in part on the discovery that factors in a biological sample from a subject can lead to an alteration in expression level of one or more genes in a disease-specific manner. Thus the activation, or repression, of the promoter of a disease-responsive gene can be used to develop reporter assays for the detection and diagnosis of diseases. In various embodiments, the invention relates to compositions comprising disease-responsive reporter constructs for use in diagnosing diseases, methods of generating the disease-responsive reporter constructs, and methods of using the disease-responsive reporter constructs to diagnose diseases and disorders.

### Compositions

In one embodiment, the invention relates to disease-responsive reporter constructs for use in methods of diagnosing a disease or disorder in a biological sample of a subject. In another embodiment, the invention relates to vectors and cells comprising a disease-responsive reporter constructs for use in methods of diagnosing a disease or disorder in a biological sample of a subject. In one embodiment, a disease-response promoter of the invention provides a nucleic acid molecule comprising at least one promoter sequence of a protein that is altered (i.e., upregulated or downregulated) upon contact with a biological sample from a subject with a disease to be detected or diagnosed, operably linked to at least one reporter marker.

In one embodiment, the disease-responsive promoter sequence is optimized for expression in a host cell. A promoter can be optimized based on the type of host cell or to optimize the signal:noise ratio for expression of the reporter gene (e.g., decrease expression in the presence of a control sample and/or increase expression in the presence of a sample from a subject having or being tested for the disease for which the disease-responsive promoter is designed to respond to.

In one embodiment, a disease-responsive promoter sequence comprises a translational initiation sequence or enhancer, such as the so-called “Kozak sequence” (Kozak, J. Cell Biol. 108: 229-41 (1989)) or “Shine-Dalgarno” sequence. In one embodiment, the promoter sequence of the disease-specific reporter construct is operably linked to a sequence encoding a reporter molecule. A reporter molecule is a molecule, including polypeptide as well as polynucleotide, expression of which in a cell confers a detectable trait to the cell. In various embodiments, reporter markers include, but are not limited to, chloramphenicol-acetyl transferase (CAT), β-galactosyltransferase, horseradish peroxidase, luciferase, NanoLuc®, alkaline phosphatase, and fluorescent proteins including, but not limited to, green fluorescent proteins (e.g. GFP, TagGFP, T-Sapphire, Azami Green, Emerald, mWasabi, mClover3), red fluorescent proteins (e.g. mRFP1, JRed, HcRedl, AsRed2, AQ143, mCherry, mRuby3, mPlum), yellow fluorescent proteins (e.g. EYFP, mBanana, mCitrine, PhiYFP, TagYFP, Topaz, Venus), orange fluorescent proteins (e.g. DsRed, Tomato, Kusabria Orange, mOrange, mTangerine, TagRFP), cyan fluorescent proteins (e.g. CFP, mTFP1, Cerulean, CyPet, AmCyanl), blue fluorescent proteins (e.g. Azurite, mtagBFP2, EBFP, EBFP2, Y66H), near-infrared fluorescent proteins (e.g. iRFP670, iRFP682, iRFP702, iRFP713 and iRFP720), infrared fluorescent proteins (e.g. IFP1.4) and photoactivatable fluorescent proteins (e.g. Kaede, Eos, IrisFP, PS-CFP).

In one embodiment, two or more reporter markers are under control of the promoter sequence of the disease-specific promoter, to provide amplification of the signal. Therefore, in various embodiments, the invention provides disease-specific reporter constructs comprising a disease-responsive promoter operably linked to nucleotide sequences encoding at least 1, at least 2, at least 3, at least 4, at least 5, or more than 5 reporter markers. In one embodiment, two or more tandem reporter markers are all the same (e.g. two tandem copies of luciferase.) Alternatively, two or more tandem reporter markers may be of different reporters.

In one embodiment, a disease-responsive reporter construct of the invention comprises one or more mRNA stabilization sequence. An mRNA stabilization sequence may alter the half-life of an mRNA molecule encoding a reporter gene and fused to the sequence such that the reading frame is maintained. In one embodiment the mRNA stabilization sequence is a polynucleotide sequence that increases the half-life of a linked mRNA. In one embodiment the mRNA stabilization sequence is a polynucleotide sequence that decreases the half-life of a linked mRNA. In one embodiment, a mRNA stabilization sequence is a poly(A) tail which protects the mRNA molecule from enzymatic degradation in the cytoplasm. In one embodiment, a mRNA stabilization sequence is a MALAT1 3′ stabilization sequence.

In one embodiment, a disease-responsive reporter construct comprises a transcription termination sequence. A typical transcriptional termination sequence includes a polyadenylation site (poly A site). In one embodiment, a poly A site is the SV40 poly A site. These sequences may be located in the disease-responsive reporter construct 3′ to a reporter gene sequence or a selection marker sequence.

In one embodiment, a disease-responsive reporter construct comprises one or more termination/stop codon(s) in one or more reading frames at the 3′ end of a reporter marker sequence or selection marker sequence, such that translations of these sequences are terminated at the stop codon(s).

Recurrent Nephrotic Syndrome (RNS) Reporter Construct

In one embodiment, a disease-response promoter designed to be responsive to a RNS of the invention provides a nucleic acid molecule comprising at least one promoter sequence of a protein whose expression is altered upon contact with a biological sample from a subject with RNS, operably linked to at least one reporter marker. In one embodiment, the RNS is rFSGS.

In one embodiment, a disease-response promoter designed to be responsive to RNS comprises a region 5′ to a gene that is upregulated in RNS. Exemplary genes that are upregulated in RNS include, but are not limited to, ABCC6, AC005682.5, AC084018.1, ADAMTS1, ADAMTS5, ASB2, BAAT, BMF, CCDC147, CCL2, CDKN1C, CDKN2B, CHAC1, CSAD, CSGALNACT1, CYP1B1, CYP24A1, DDIT3, DTX3, FBXLS, GREM1, GSDMB, HCPS, HLA-DMB, HSD17B7P2, IGFBP3, IGFBP5, IL-11, IL-1A, IL-1β, KCTD19, KCTD4, KRT23, KRTAP1-1, KRTAP2-3, LINC00263, MAP3K7CL, NEAT1, NPPB, NR1D2, PLA2G4C, PLK1S1, PTGIS, RAPGEF3, RNF207, RP11-16112.1, RP11-336N8.4, RP5-1021120.2, RPL13AP20, RRNAD1, RUSC1-AS1, SEMA3A, SLC22A3, SLC25A27, SLC7A14, SMOC1, SMPD3, SUSD4, TMEM86A, TNF, TNFSF10, TRIM45, TRPC4, WDR66, XXbac-BPG246D15.8. In one embodiment, the disease-response promoter comprises a region 5′ to at least one of BMF, KRTAP2-3, IL-1β, SLC7A14, ASB2, CYP1B1, KRT23, NPPB, and ADAMTS5. In one embodiment, the disease-response promoter comprises a region 5′ to at least one of IGFBP3, IL-1β and BMF.

In one embodiment, a disease-response promoter designed to be responsive to RNS comprises a region 5′ to a gene that is downregulated in RNS. Exemplary genes that are downregulated in RNS include, but are not limited to, TMEM2, KIAA0247, RERE, PDXK, TAP1, CARHSP1, NEDD9, TAPSAR1, CPD and CNDP2.

In one embodiment, a disease-response promoter designed to be responsive to RNS comprises the nucleotide sequence of at least 50 nucleotides, at least 250 nucleotides, at least 500 nucleotides, at least 750 nucleotides, at least 1000 nucleotides, at least 1500 nucleotides, at least 2000 nucleotides, at least 2500 nucleotides, at least 3000 nucleotides, at least 3500 nucleotides, at least 4000 nucleotides, at least 4500 nucleotides, at least 5000 nucleotides or more than 5000 nucleotides immediately upstream of the translational start codon of at least one of ABCC6, AC005682.5, AC084018.1, ADAMTS1, ADAMTS5, ASB2, BAAT, BMF, CCDC147, CCL2, CDKN1C, CDKN2B, CHAC1, CSAD, CSGALNACT1, CYP1B1, CYP24A1, DDIT3, DTX3, FBXLS, GREM1, GSDMB, HCPS, HLA-DMB, HSD17B7P2, IGFBP3, IGFBP5, IL-11, IL-1A, IL-1β, KCTD19, KCTD4, KRT23, KRTAP1-1, KRTAP2-3, LINC00263, MAP3K7CL, NEAT1, NPPB, NR1D2, PLA2G4C, PLK1S1, PTGIS, RAPGEF3, RNF207, RP11-16112.1, RP11-336N8.4, RP5-1021120.2, RPL13AP20, RRNAD1, RUSC1-AS1, SEMA3A, SLC22A3, SLC25A27, SLC7A14, SMOC1, SMPD3, SUSD4, TMEM86A, TNF, TNFSF10, TRIM45, TRPC4, WDR66, XXbac-BPG246D15.8, TMEM2, KIAA0247, RERE, PDXK, TAP1, CARHSP1, NEDD9, TAPSAR1, CPD and CNDP2.

In one embodiment, a disease-response promoter designed to be responsive to RNS comprises the nucleotide sequence of about 4000 nucleotides immediately upstream of the translational start codon of BMF. In one embodiment, the promoter of BMF is amplified using the primers as set forth in SEQ ID NO:1 and SEQ ID NO:2. In one embodiment, the promoter of BMF comprises the sequence set forth in SEQ ID NO:15, or a variant or fragment thereof. In one embodiment, the promoter sequence comprises the nucleotide sequence of about 4000 nucleotides immediately upstream of the translational start codon of IL-1β. In one embodiment, the promoter of IL-1β is amplified using the primers as set forth in SEQ ID NO:3 and SEQ ID NO:4. In one embodiment, the promoter of IL-1β comprises the sequence set forth in SEQ ID NO:16, or a variant or fragment thereof. In one embodiment, the promoter sequence comprises the nucleotide sequence of about 3000 nucleotides immediately upstream of the translational start codon of IGFBP3. In one embodiment, the promoter of IGFBP3 is amplified using the primers as set forth in SEQ ID NO:5 and SEQ ID NO:6. In one embodiment, the promoter of IGFBP3 comprises the sequence set forth in SEQ ID NO:17, or a variant or fragment thereof.

Variants of a disease-response promoter can also be used in reporter constructs of the invention. In one embodiment, a variant comprising at least 60%, at least 65%, at least 70%, at least 75%, at least 80%, at least 85%, at least 90%, at least 95%, at least 96%, at least 97%, at least 98%, or at least 99% sequence identity to SEQ ID NO:15, SEQ ID NO:16 or SEQ ID NO:17 is operably linked to a reporter molecule.

Fragments of a disease-response promoter can also be used in reporter constructs of the invention. In one embodiment, a fragment comprising at least 60%, at least 65%, at least 70%, at least 75%, at least 80%, at least 85%, at least 90%, at least 95%, at least 96%, at least 97%, at least 98%, or at least 99% of the full length of SEQ ID NO:15, SEQ ID NO:16 or SEQ ID NO:17 is operably linked to a reporter molecule.

Variants of fragments of SEQ ID NO:15, SEQ ID NO:16 or SEQ ID NO:17 can be provided. Such variants can comprise amino acid sequences having at least 60%, at least 65%, at least 70%, at least 75%, at least 80%, at least 85%, at least 90%, at least 95%, at least 96%, at least 97%, at least 98%, or at least 99% of the full length protein sequence having 60%, at least 65%, at least 70%, at least 75%, at least 80%, at least 85%, at least 90%, at least 95%, at least 96%, at least 97%, at least 98%, or at least 99% or greater identity to SEQ ID NO:15, SEQ ID NO:16 or SEQ ID NO:17.

Vector

The present invention also includes a vector in which the disease-responsive reporter construct of the present invention is inserted. The art is replete with suitable vectors that are useful in the present invention.

In brief summary, the expression of a reporter construct is typically achieved by operably linking a nucleic acid sequence comprising a promoter to a nucleic acid sequence encoding a reporter molecule or portions thereof, and incorporating the construct into an expression vector. In one embodiment, the vectors to be used are suitable for replication and, optionally, integration in eukaryotic cells. Typical vectors contain transcription and translation terminators, initiation sequences, and other regulatory sequences useful for regulation of the expression of the desired nucleic acid sequence.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method

In certain embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the reporter molecule and expression control sequences that act in trans or at a distance to control the expression of the reporter molecule. Expression control sequences include appropriate transcription initiation, termination, and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. All of the above-described functional elements can be used in any combination to produce a suitable disease-responsive reporter vector.

In one embodiment, a disease-responsive reporter vector comprises an origin of replication capable of initiating DNA synthesis in a suitable host cell. In one embodiment, the origin of replication is selected based on the type of host cell. For instance, it can be eukaryotic (e.g., yeast) or prokaryotic (e.g., bacterial) or a suitable viral origin of replication may be used.

In one embodiment, a disease-responsive reporter vector comprises a selection marker gene to facilitate identification and selection of expressing cells from the population of cells sought to be transfected or infected through viral vectors. In other aspects, the selectable marker may be carried on a separate piece of DNA and used in a co-transfection procedure. Selectable marker genes may be flanked with appropriate regulatory sequences to enable expression in the host cells.

A selection marker sequence can be used to eliminate host cells in which the disease-responsive reporter vector has not been properly transfected. A selection marker sequence can be a positive selection marker or negative selection marker. Positive selection markers permit the selection for cells in which the gene product of the marker is expressed. This generally comprises contacting cells with an appropriate agent that, but for the expression of the positive selection marker, kills or otherwise selects against the cells. For suitable positive and negative selection markers, see Table I in U.S. Pat. No. 5,464,764.

Examples of selection markers also include, but are not limited to, proteins conferring resistance to compounds such as antibiotics, proteins conferring the ability to grow on selected substrates, proteins that produce detectable signals such as luminescence, catalytic RNAs and antisense RNAs. A wide variety of such markers are known and available, including, for example, a Zeocin™ resistance marker, a blasticidin resistance marker, a neomycin resistance (neo) marker (Southern & Berg, J. Mol. Appl. Genet. 1: 327-41 (1982)), a puromycin (puro) resistance marker; a hygromycin resistance (hyg) marker (Te Riele et al., Nature 348:649-651 (1990)), thymidine kinase (tk), hypoxanthine phosphoribosyltransferase (hprt), and the bacterial guanine/xanthine phosphoribosyltransferase (gpt), which permits growth on MAX (mycophenolic acid, adenine, and xanthine) medium. See Song et al., Proc. Nat'l Acad. Sci. U.S.A. 84:6820-6824 (1987). Other selection markers include histidinol-dehydrogenase, chloramphenicol-acetyl transferase (CAT), dihydrofolate reductase (DHFR), β-galactosyltransferase and fluorescent proteins such as GFP.

Expression of a fluorescent protein can be detected using a fluorescent activated cell sorter (FACS). Expression of β-galactosyltransferase also can be sorted by FACS, coupled with staining of living cells with a suitable substrate for β-galactosidase. A selection marker also may be a cell-substrate adhesion molecule, such as integrins, which normally are not expressed by the host cell. In one embodiment, the cell selection marker is of mammalian origin, for example, thymidine kinase, aminoglycoside phosphotransferase, asparagine synthetase, adenosine deaminase or metallothionien. In one embodiment, the cell selection marker can be neomycin phosphotransferase, hygromycin phosphotransferase or puromycin phosphotransferase, which confer resistance to G418, hygromycin and puromycin, respectively.

Suitable prokaryotic and/or bacterial selection markers include proteins providing resistance to antibiotics, such as kanamycin, tetracycline, and ampicillin. In one embodiment, a bacterial selection marker includes a protein capable of conferring selectable traits to both a prokaryotic host cell and a mammalian target cell.

Negative selection markers permit the selection against cells in which the gene product of the marker is expressed. In some embodiments, the presence of appropriate agents causes cells that express “negative selection markers” to be killed or otherwise selected against. Alternatively, the expression of negative selection markers alone kills or selects against the cells.

Such negative selection markers include a polypeptide or a polynucleotide that, upon expression in a cell, allows for negative selection of the cell. Illustrative of suitable negative selection markers are (i) herpes simplex virusthymidine kinase (HSV-TK) marker, for negative selection in the presence of any of the nucleoside analogs acyclovir, gancyclovir, and 5-fluoroiodoamino-Uracil (FIAU), (ii) various toxin proteins such as the diphtheria toxin, the tetanus toxin, the cholera toxin and the pertussis toxin, (iii) hypoxanthine-guanine phosphoribosyl transferase (HPRT), for negative selection in the presence of 6-thioguanine, (iv) activators of apoptosis, or programmed cell death, such as the bcl2-binding protein (BAX), (v) the cytidine deaminase (codA) gene of E. coli. and (vi) phosphotidyl choline phospholipase D. In one embodiment, the negative selection marker requires host genotype modification (e.g. ccdB, tolC, thyA, rpsl and thymidine kinases.)

In accordance with the present invention, the selection marker usually is selected based on the type of the cell undergoing selection. For instance, it can be eukaryotic (e.g., yeast), prokaryotic (e.g., bacterial) or viral. In such an embodiment, the selection marker sequence is operably linked to a promoter that is suited for that type of cell.

### Genome Editing Compositions

In one embodiment, the invention provides for integration of the reporter system of the invention into a host cell through use of a genome editing system. A series of programmable nuclease-based genome editing technologies have developed (see for example, Hsu et al., Cell 157, Jun. 5, 2014 1262-1278), including, but not limited to, meganucleases, zinc finger nucleases (ZFNs), transcription activator-like effector-based nucleases (TALENs) and CRISPR-Cas9 systems (see e.g. Platt et al., Cell 159(2), 440-455 (2014); Shalem et al., Science 3 84-87 (2014); and Le Cong et al., Science 339, 819 (2013)) or alternative CRISPR systems. Genome editing systems have a wide variety of utilities including modifying (e.g., deleting, inserting, translocating, inactivating, activating, repressing, altering methylation, transferring specific moieties) a target polynucleotide in a multiplicity of cell types. In one embodiment, a CRISPR-Cas9 system is used to integrate the reporter constructs of the invention into a host genome. The CRISPR-Cas9 system can include at least one guide RNA (gRNA) targeted to a target nucleic acid sequence, and a CRISPR-associated (Cas) peptide form a complex to induce insertion of the reporter constructs at the targeted nucleic acid sequence.

In one embodiment, the target polynucleotide is a DNA molecule. DNA molecules include, but are not limited to, genomic DNA molecules, extrachromosomal DNA molecules, conjugative plasmids and exogenous DNA molecules.

In general, “CRISPR-Cas9 system” or “CRISPR system” refers collectively to transcripts and other elements involved in the expression of or directing the activity of CRISPR-associated (“Cas”) genes, including sequences encoding a Cas9 gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system are derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system).

In some embodiments, the site of reporter integration is determined by the CRISPR-Cas9 system guide RNA. In general, a “CRISPR-Cas9 guide RNA” or “guide RNA” refers to an RNA that directs sequence-specific binding of a CRISPR complex to the target sequence. Typically, a guide RNA comprises (i) a guide sequence that has sufficient complementarity with a target polynucleotide sequence to hybridize with the target sequence and (ii) a trans-activating cr (tracr) mate sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In the context of formation of a CRISPR complex, a “target sequence” or “a sequence of a target DNA” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides or DNA/RNA hybrid polynucleotides. In some embodiments, a target sequence is located in the nucleus or cytoplasm of a cell. In some embodiments, the target sequence may be within an organelle of a eukaryotic cell, for example, mitochondrion or chloroplast.

In some embodiments, the CRISPR-Cas9 domain comprises a Cas9 protein. Non-limiting examples of Cas9 proteins include Cast, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, orthologs thereof, or modified versions thereof. In some embodiments, the Cas9 protein has DNA or RNA cleavage activity. In some embodiments, the Cas9 protein directs cleavage of one or both strands of a nucleic acid molecule at the location of a target sequence, such as within the target sequence and/or within the complement of the target sequence. In some embodiments, the Cas9 protein directs cleavage of one or both strands within about 1, 2, 3, 4, 5, 6, 7, 8, 9, 10, 15, 20, 25, 50, 100, 200, 500, or more base pairs from the first or last nucleotide of a target sequence.

Cells

In one embodiment, the invention relates to cells or cell lines containing a disease-responsive reporter construct of the invention. Methods of introducing and expressing genes in a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). In one embodiment, the method of introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

In one embodiment, the present invention provides a cell or population of cells modified to comprise a disease-responsive reporter construct of the invention. In one embodiment, the cells are prokaryotic cells. In one embodiment, cells are eukaryotic cells. In one embodiment, a cell is a mammalian cell, such as a murine or human cell. The target cell may be a somatic cell or a germ cell. The germ cell may be a stem cell, such as embryonic stem cells (ES cells), including murine embryonic stem cells. The target cell may be podocyte or a podocyte precursor cell.

The target cell may be chosen from commercially available mammalian cell lines. The target cell may be a primary cell isolated from a subject. A target cell may be any type of diseased cell, including cells with abnormal phenotypes that can be identified using biological or biochemical assays. For instance, the diseased cell may be a tumor cell. In one embodiment, a cell may be an HEK293 cell.

The cells of the invention and cells derived therefrom can be derived from, inter alia, humans, primates, rodents and birds. In one embodiment, the cells of the invention are derived from mammals, especially mice, rats and humans. In one embodiment, cells may be either wild-type or genetically modified cells.

The cells of the present invention, whether grown in suspension or as adherent cell cultures, are grown in contact with culture media.

In one embodiment, culture media used in the present invention comprises a basal medium, optionally supplemented with additional components. Basal medium is a medium that supplies essential sources of carbon and/or vitamins and/or minerals for the cells. The basal medium is generally free of protein and incapable on its own of supporting self-renewal/symmetrical division of the cells. Media formulations that support the growth of cells include, but are not limited to, Minimum Essential Medium Eagle, ADC-1, LPM (bovine serum albumin-free), F10 (HAM), F12 (HAM), DCCM1, DCCM2, RPMI 1640, BGJ Medium (with and without Fitton-Jackson Modification), Basal Medium Eagle (BME—with the addition of Earle's salt base), Dulbecco's Modified Eagle Medium (DMEM-without serum), Yamane, IMEM-20, Glasgow Modification Eagle Medium (GMEM), Leibovitz L-15 Medium, McCoy's 5A Medium, Medium M199 (M199E—with Earle's salt base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA with nonessential amino acids), and the like.

It is further recognized that additional components may be added to the culture medium. Such components include, but are not limited to, antibiotics, antimycotics, albumin, growth factors, amino acids, and other components known to the art for the culture of cells. Antibiotics which can be added into the medium include, but are not limited to, penicillin and streptomycin. The concentration of penicillin in the culture medium is about 10 to about 200 units per ml. The concentration of streptomycin in the culture medium is about 10 to about 200 μg/ml. However, the invention should in no way be construed to be limited to any one medium for culturing the cells of the invention. Rather, any media capable of supporting the cells of the invention in tissue culture may be used.

Typical substrates for culture of the cells in all aspects of the invention are culture surfaces recognized in this field as useful for cell culture, and these include surfaces of plastics, metal, composites, though commonly a surface such as a plastic tissue culture plate, widely commercially available, is used. Such plates are often a few centimeters in diameter. For scale up, this type of plate can be used at much larger diameters and many repeat plate units used. For high throughput assays multi-well plates, having 6, 12, 24, 48, 96 or more wells can be used.

The culture surface may further comprise a cell adhesion protein, usually coated onto the surface. Receptors or other molecules present on the cells bind to the protein or other cell culture substrate and this promotes adhesion to the surface and promotes growth. In certain embodiments, the cultures of the invention are adherent cultures, i.e. the cells are attached to a substrate.

### Methods for Developing Disease-Specific Reporter Constructs

In one embodiment, the invention provides methods for developing disease-responsive reporter constructs for use in the methods of the invention. In one embodiment, the method includes a) contacting cells with a biological sample from a subject having a disease or disorder for which a disease-responsive reporter construct is desired, b) isolating RNA from the cells, c) performing an analysis on the isolated RNA to identify candidate genes whose expression level was altered in the presence of the biological sample from the subject having the disease or disorder, d) identifying the promoter region of one or more candidate genes, and e) constructing a disease-specific reporter construct comprising the promoter of the candidate gene operably linked to a nucleotide sequence encoding one or more reporter markers.

In one embodiment, the biological sample is a fluid sample (e.g., a blood sample, a serum sample, a plasma sample, a saliva sample, or a urine sample). In one embodiment, the biological sample is a tissue sample (e.g., a biopsy sample). In one embodiment, the sample is treated or prepared prior to contact with the cells. Methods of treating or preparing a biological sample include, but are not limited to, centrifugation, homogenization, freezing, or other techniques that are known to those of skill in the art for preparing a biological sample. The biological sample can be obtained by appropriate methods, such as, by way of examples, blood draw, fluid draw, or saliva collection.

The isolated RNA can be assayed by any appropriate method to identify candidate genes whose expression level was altered in the presence of the biological sample from the subject. Methods for assaying the level of one or more RNA molecule are well known to those of skill in the art and include, but are not limited to, high-throughput sequencing protocols such as RNA-seq.

### Diagnostic Assay

In some embodiments, the invention relates to diagnostic assays to diagnose a disease or disorder in a subject in need thereof using the disease-responsive reporter constructs of the invention. In one embodiment, the assay includes contacting a cell comprising disease-responsive reporter construct with a test sample from a subject, measuring an increase or decrease in the expression of the reporter marker, and diagnosing the subject as having the disease or disorder for which the disease-responsive reporter construct is responsive to based on an alteration in the expression level of the reporter marker. The diagnostic assays of the invention are not limited to a specific disease or disorder, but may be used to diagnose the presence, risk, or stage of any disease or disorder for which a disease-responsive reporter construct is designed to be responsive to.

In various embodiments, the test sample is a biological sample (e.g., fluid, tissue, cell, cellular component, etc.) of the subject. In one embodiment, a biological sample is a fluid sample. A biological sample can be obtained by appropriate methods, such as, by way of examples, blood draw, fluid draw, or saliva collection. A biological sample can be used as the test sample; alternatively, a biological sample can be processed, and the processed biological sample can then be used as the test sample. For example, in various embodiments, a serum sample may be processed to generate a plasma sample, which is then used as the test sample.

The results of the diagnostic assay can be used alone, or in combination with other information from the subject, or other information from the biological sample obtained from the subject.

In the assay methods of the invention, a test biological sample from a subject is assessed for the ability to induce expression of a reporter construct. The level of expression of the reporter construct can be determined by assessing the amount of a polypeptide gene product produced, the amount of a mRNA gene product produced, the amount of activity of a gene product produced, the amount of binding activity of a gene produce produced, or a combination thereof.

In various embodiments, to determine whether a biological sample from a subject results in an increase in expression of a reporter construct, the level of expression of the reporter construct is compared with the level of at least one comparator control, such as a positive control, a negative control, a historical control, a historical norm, or the level of another reference molecule in the biological sample.

In various embodiments of the assays of the invention, the level of expression is determined to be elevated when the level of expression is increased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator control.

In various embodiments of the assays of the invention, the level of expression is determined to be elevated when the level of expression is increased by at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.5 fold, at least 4.0 fold, at least 4.5 fold, at least 5.0 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, when compared with a comparator control.

The disease-specific reporter constructs of the invention can be used for diagnosing a disease using any appropriate method for detecting the level of expression of a reporter marker. Appropriate methods include both high-throughput and low-throughput methods.

In various embodiments of the invention, methods of measuring the level of expression of a reporter marker include, but are not limited to, an immunochromatography assay, an immunodot assay, a luminescence assay, an ELISA assay, an ELISPOT assay, a protein microarray assay, a ligand-receptor binding assay, displacement of a ligand from a receptor assay, displacement of a ligand from a shared receptor assay, an immunostaining assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

### RNS Diagnostic Assays

In one embodiment, the invention provides a diagnostic assay for diagnosing a RNS in a subject. In one embodiment, the assay includes contacting a cell comprising an RNS-responsive reporter construct with a biological sample from a subject, and measuring an increase or decrease in the expression of the reporter marker. In one embodiment, the RNS diagnostic assay can be used to identify a subject as having or at risk of RNS. In one embodiment, the RNS diagnostic assay can be used to differentiate between a non-recurrent nephrotic syndrome and recurrent nephrotic syndrome. In one embodiment, the RNS diagnostic assay can be used to differentiate between RNS and other nephropathies, including, but not limited to, membranous nephropathy (MN) and membranous glomerulonephritis (MGN). In one embodiment, the RNS diagnostic assay can be used to identify a subject as having an increased risk of kidney graft rejection.

In one embodiment, the RNS is rFSGS. Therefore, in one embodiment, the assay includes contacting a cell comprising an rFSGS-responsive reporter construct with a biological sample from a subject, and measuring an increase or decrease in the expression of the reporter marker. In one embodiment, the rFSGS diagnostic assay can be used to identify a subject as having or at risk of rFSGS. In one embodiment, the rFSGS diagnostic assay can be used to differentiate between FSGS and rFSGS. In one embodiment, the rFSGS diagnostic assay can be used to differentiate between rFSGS and other nephropathies, including, but not limited to, membranous nephropathy (MN) and membranous glomerulonephritis (MGN). In one embodiment, the rFSGS diagnostic assay can be used to identify a subject as having an increased risk of kidney graft rejection.

In various embodiments, the subject is a human subject, and may be of any race, sex and age. Representative subjects include those who are suspected of having RNS, FSGS or another nephropathy, those who have been diagnosed with RNS, rFSGS, FSGS or another nephropathy, those who have developed RNS, rFSGS, FSGS or another nephropathy, those who at risk of developing RNS, rFSGS, FSGS or another nephropathy those who have been diagnosed with a kidney disease or disorder, those who have kidney failure, those who are undergoing a treatment for a kidney disease or disorder and those who are in need of a kidney transplant.

### Methods of Treatment

In certain embodiments, the method of the present invention comprises effecting a therapy based on the assessment of risk for a disease or disorder. For example, in certain embodiments, the detection of altered expression of a disease-responsive reporter construct, indicates that the subject may have or be at increased risk of the disease or disorder to which the reporter construct is designed to be responsive. In one embodiment, a treatment is administered based on the results of the diagnostic assay. For example, in one embodiment, the disease is rFSGS, and the method comprises administering plasmapheresis, calcineurin inhibitors (e.g, cyclosporine and tacrolimus) or a combination thereof to the subject when it is determined that the subject has or is at risk of rFSGS based on an alteration in expression level of an rFSGS-responsive reporter construct as compared to a comparator control. In one embodiment, effecting a therapy comprises performing a medical procedure on a subject. In one embodiment, a medical procedure comprises plasmapheresis. The subject may undergo a single or multiple medical procedures (e.g., plasmapheresis and hemodialysis) or a single or multiple rounds of a medical procedure. In one embodiment, the method may comprise performing a kidney transplant, or performing one or more additional diagnostic assays on the subject when it is determined that the subject is not at increased risk of rFSGS.

Measurement of expression of the disease-responsive reporter construct also allows for the course of treatment of a disease to be monitored. The effectiveness of a treatment regimen for a disease or disorder can be monitored by detecting expression of the disease-responsive reporter construct when contacted with multiple samples obtained from a subject over time and comparing the amount of expression detected. For example, a first sample can be obtained prior to the subject receiving treatment and one or more subsequent samples are taken after or during treatment of the subject. Changes in reporter construct expression levels across the samples may provide an indication as to the effectiveness of the therapy.

In one embodiment, effecting a therapy comprises administering an effective amount of a disease-modulating drug to the subject. The subject may be treated with one or more drugs until the subject shows a changed level or improvement in one or more clinical parameter associated with the disease.

Any drug or combination of drugs or procedures disclosed herein may be administered to a subject to diagnosed as having or at risk of a disease or disorder. The drug, therapy or combination thereof herein can be administered according to any number of formulations and/or treatment regimens, often according to various known formulations and/or treatment regimens in the art. An appropriate treatment regimen will be readily apparent to the skilled artisan and will depend upon any number of factors, such as, but not limited to, the subject's age, sex, weight, health, and the route of administration and formulation used.

### Kits

The present invention also pertains to kits useful in the methods of the invention. Such kits comprise various combinations of components useful in any of the methods described elsewhere herein, including for example, disease-responsive reporter constructs, vectors or cell lines, and instructional material. In various embodiments, to determine whether the level of a disease-responsive reporter construct is altered upon contact with a biological sample obtained from a subject, the level of expression of a disease-responsive reporter construct is compared with the level of at least one comparator control contained in the kit, such as a positive control, a negative control, a historical control, a historical norm, or the level of a reporter construct that is not disease-responsive (e.g., a control reporter construct.)

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the exemplary embodiments of the present invention, and are not to be construed as limiting in any way the remainder of the disclosure.

### Example 1: A Novel Cell-Based Assay for Diagnosing Recurrent Focal and Segmental Glomerulosclerosis (rFSGS)

In this invention, a novel podocyte cell-based approach is described, which can be easily adapted commercially and has superior detection efficiency in diagnosing rFSGS patients. This is the first study describing a reporter-based system to detect a glomerular disease. While previous approaches have primarily relied on directly analyzing human plasma, profiling of human podocytes that were treated with rFSGS or control patient plasma was performed. Through comparisons with controls, rFSGS responsive genes were selected, whose promoter regions were used to construct three reporter cell lines (BMF, IL1β and IGFBP3) that specifically responded to plasma from rFSGS patients. Importantly, a control cell line (LAMP3) derived from the promoter of a non-responsive rFSGS gene did not respond in this assay. Most notably, the assay responded to patient plasma that was frozen for years, further indicating the feasibility of this approach in a commercial setup that may require transportation of plasma to various centers where diagnosis can be made.

Since a variable response was expected due to patient diversity and possibly different circulating factors, multiple promoters for constructing cell lines that are the backbone of this assay were selected. The statistical analyses further confirm the specificity of these multiple reporter cell lines, which individually provide more than 80% specificity for diagnosis. While only three gene promoters were selected to prove the effectiveness of the concept, it is possible that additional gene promoters can be identified and included in this assay to further strengthen the specificity of this assay. It is to be noted that the magnitude of response in this luciferase-based assay was 1.5-2 fold, which was maintained across the entire cohort tested. Additionally, since this assay was shown to be independent of the host cell line, to further increase the assay response, multiple cell lines can be screened to select a cell line that will generate maximal response from these promoters. This also suggests that further optimization may be required prior to determining its commercial value.

This invention provides strong support for the effectiveness of the approach and concept. Strikingly, the assay was able to differentiate between rFSGS from non-recurrent FSGS, a finding of major importance for the optimal management of these patients. In conclusion, a novel concept is presented that was used to develop a cost-effective and technically simple assay to specifically diagnose rFSGS patients. Once developed, this assay can be performed on all patients with suspected FSGS and nephrotic range proteinuria. Ultimately, this assay will not only be able to predict recurrence of FSGS in allografts but also constitute a non-invasive tool for diagnosis of primary FSGS in native kidneys caused by permeability factor(s).

The assay is based on the known fact that the treatment of cultured human podocytes with human FSGS patient plasma induces molecular events that result in significant alterations in the actin cytoskeleton partially mimicking the disease processes.

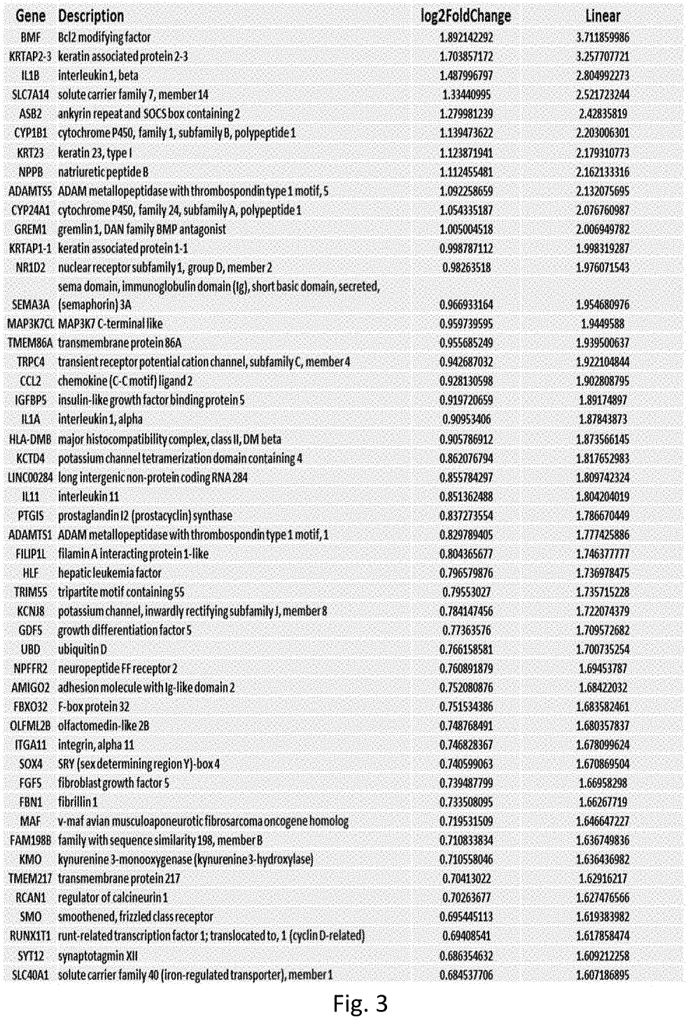

In this invention, it is evident that treatment of podocytes with human FSGS patient plasma induces a gene expression profile in podocytes that establishes a molecular pattern of the disease. Indeed, profiling studies on podocytes treated with plasma from various FSGS patients identified a set of genes that represent signaling pathways including the JNK, TRP, TNF, MAPK: Oxidative Stress pathway, NFκB pathway, Estrogen Metabolism pathways etc. that may contribute to a disease phenotype. Among the few upregulated candidate genes from profiling studies (Table 1 and FIG. 3), BMF (BC12 Modifying Factor) and IL-1β were selected as candidate FSGS responsive genes and their promoters were used to develop Luciferase reporter-based stable podocyte cell lines. Without being bound by a particular theory, these cell lines were constructed with the notion that upon contact with rFSGS patient plasma, the cloned promoter will be activated, specifically driving the expression of luciferase protein. The change in expression is measured by luminometer. In addition, to the podocyte cell lines, chimeric human embryonic kidney cell (HEK) lines with these promoters were also generated and tested in the assay. All these cell lines were tested for their ability to respond to rFSGS plasma as described below.

The robustness of this assay was tested by screening various FSGS and non-FSGS patient plasma procured from different centers within the USA, and the potential as a diagnostic tool for detecting rFSGS was validated. Overall, results obtained from this invention provide the basis for development of a noninvasive diagnostic tool.

Although the approach is simple, selection of appropriate target gene promoter sequence is crucial for the success of this type of investigative technique. Therefore, extensive bioinformatics analysis was performed to identify the correct promoter sequence and then standardized the ‘not so easy to amplify’ a 4 kb long promoter region which is full of repeats and GC rich. The promoter sequence was amplified and cloned into the reporter vector and its fidelity was confirmed by sequencing.

The materials and methods used in this example are now described.

Cell Culture and Immunofluorescence Microscopy and Antibodies

Human podocytes were cultured using RPMI medium supplemented with 10% FBS, 2 g/liter of NaHCO₃, insulin-transferrin-selenium (ITS) supplement and 200 units/ml penicillin and streptomycin as described previously at 33° C. and 5% CO₂ (Wagner, M. C. et al., 2008, J Biol Chem., 283(51): p. 35579-89). Podocytes were differentiated by thermoswitching to 37° C. and removing the ITS supplement from the RPMI media as described previously (Wagner, M. C. et al., 2008, J Biol Chem., 283(51): p. 35579-89). Podocytes were grown to 80-90% confluency on glass coverslips coated with collagen. Podocytes were serum starved over-night and treated with 4% patient plasma in serum free RPMI medium for 12-16 hours. The plasma for all the listed patients were collected during plasmapheresis. The patients underwent plasmapheresis as soon as they were diagnosed with heavy proteinuria, and the plasma samples were collected immediately before the plasmapheresis was initiated. Podocytes were fixed with 4% paraformaldehyde and immunostained with Alexa-Fluor-488 phalloidin and DAPI. Fluorescence microscopy was performed using Leica Microscope, DMI 4000B and ImageJ software was used to finalize these images. The human embryonic kidney (HEK293) and COST cells were cultured in DMEM supplemented with 10% FBS and 1% penicillin and streptomycin. CD63 antibody was procured commercially from ThermoFisher Scientific, Cat No: 10628D.

Candidate Gene Identification

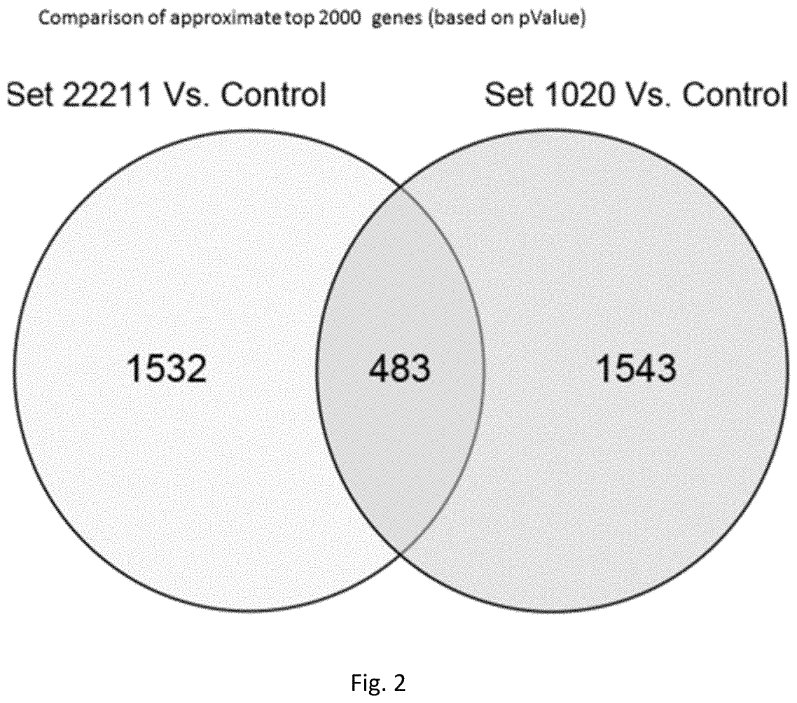

Analysis of RNA Seq data from multiple rFSGS patient samples identified genes commonly upregulated in rFSGS (FIG. 2, FIG. 3 and Table 1).

TABLE 1
List of few top upregulated genes from RNA Seq data

| Sample Name: PJ | Sample Name: DD | Sample Name: 1020 |
|---|---|---|
| SLC22A3 | RP11-336N8.4 | BMF |
| RAPGEF3 | IGFBP3 | KRTAP2-3 |
| SUSD4 | BAAT | IL1β |
| DTX3 | WDR66 | SLC7A14 |
| IGFBP3 | CDKN1C | ASB2 |
| AC084018.1 | RP5-1021I20.2 | CYP1B1 |
| BMF | SMOC1 | KRT23 |
| NEAT1 | CHAC1 | NPPB |
| ABCC6 | CDKN2B | ADAMTS5 |
| RUSC1-AS1 | CSGALNACT1 | CYP24A1 |
| FBXL5 | HCP5 | GREM1 |
| RNF207 | TNF | KRTAP1-1 |
| TRIM45 | LINC00263 | NR1D2 |
| SLC25A27 | HSD17B7P2 | SEMA3A |
| GSDMB | DDIT3 | MAP3K7CL |
| TNFSF10 | CCDC147 | TMEM86A |
| CCDC147 | RPL13AP20 | TRPC4 |
| PLK1S1 | KCTD19 | CCL2 |
| CSAD | AC005682.5 | IGFBP5 |
| | SMPD3 | IL1A |
| | PLA2G4C | HLA-DMB |
| | IL11 | KCTD4 |
| | XXbac-BPG246D15.8 | IL11 |
| | RP11-161I2.1 | PTGIS |
| | RRNAD1 | ADAMTS1 |

mRNA Profiling of Plasma Treated Podocytes

RNA isolated from the human podocytes treated with control (one control) and two rFSGS patients (rF A and rF D) plasma were subjected to RNA Seq (mRNA profiling). RNA samples were isolated from independent experiments performed in triplicate. Quantitative and qualitative analyses of RNA was performed using Nanodrop, agarose gel electrophoresis and Agilent 2100 at Novogen and processed for RNA Seq. Briefly, library construction was performed using poly-T oligo-attached magnetic beads. After qualitative and quantitative (library effective concentration >2 nM) assessment of appropriate libraries were fed into HiSeq/MiSeq Illumina platform for the RNA sequencing. Downstream bioinformatics analyses were performed by using a combination of programs including Bowtie2, Tophat2, HTseq, Cufflink and the wrapped scripts at Novagen. Reference genome and gene model annotation files were acquired from NCBI/UCSC/Ensemble genome browser. Indexes of the reference genome were built using Bowtie v2.0.6 and paired-end clean reads were aligned to the reference genome using TopHat v2.0.9. Gene expression level quantification was done using HTSeq v0.6.1 and for each gene read numbers counts were mapped, whereas, Reads Per Kilobase of exon model (RPKM) of each gene was calculated based on the length of the gene and expression levels were analyzed (Mortazavi, A. et al., 2008, Nat Methods., 5(7): p. 621-628). DESeq2 R package (2_1.6.3) database was used for the differential expression (DE) analyses. The resulting P-values were adjusted using the Benjamini and Hochberg's approach and adjusted P-value <0.05 found by DESeq2 were assigned as DE. Analyses of DE of two conditions was performed using the DEGSeq R package (1.12.0). The P-values were also adjusted using Benjamini & Hochberg method. Corrected P-value of 0.005 and log 2 (Fold change) of 1 were set as threshold for significantly differential expression.

Identification of Candidate Gene Promoters, Cloning and Generation of Stable Podocytes Cell Lines

Three upregulated candidate genes BMF (BC12 modifying factor), IL-1β and IGFBP3 (Insulin like growth factor binding protein 3), which are also involved in cellular apoptosis, were selected from the RNA-Seq data (Geo submission # GSE117669; the top 10 upregulated and downregulated genes are shown in Table 2). LAMP3 gene promoter was chosen as a negative experimental control since it was not upregulated in the profiling data. The promoter regions (2.5 to 4 kb upstream of transcription start site) for these genes were PCR amplified using primers listed in Table 3 and cloned upstream of a luciferase reporter ORF in a promoter less vector pGL4.20Luc/Puro (Promega) (Shirakawa, F. et al., 1993, Mol Cell Biol., 13(3):1332-1344; Zhang, Y. et al., 2006, Cell Death Differ., 13(1):129-140). Promoter sequence were obtained from ensemble database and the promoter analyses software Proscan programme v1.7 (https://www-bimas.cit.nih.gov/cgi-bin/molbio/proscan) was used to confirm the sequence characteristic such as regulatory elements of a promoter like TATA box, transcription factor binding sites etc. Amplified promoter sequences were obtained after extensive PCR optimization and further confirmed by restriction digestion and sequencing. IL1β gene promoter was cloned at NheI/BglII sites (primers 2F/R), whereas BMF and IGFBP3 promoters were cloned at NheI/HindIII sites (primers 1F/R and primers 3F/R respectively) and LAMP3 at NheI/EcoRV sites (primers 4F/R). Stable podocyte cell lines expressing luciferase constructs were generated by transfecting these constructs in cultured podocytes using Lipofectamine-2000 (Thermo Scientific) followed by selection with puromycin. Control stable podocyte cell line was also prepared using blank luciferase vector. In addition to the podocyte cell lines, chimeric human embryonic kidney (HEK293) and COS7 cell lines were made with the promoters of BMF and IL1β.

TABLE 2
Top 10 upregulated and downregulated genes from the RNA-Seq
analyses of patient rF A.

| Symbol | log2Fold Change | padj |
|---|---|---|
| Upregulated Genes: | | |
| BMF | 1.892142292 | 2.6822E−56 |
| KRTAP2-3 | 1.703857172 | 6.07951E−59 |
| IL1B | 1.487996797 | 7.63113E−22 |
| SLC7A14 | 1.33440995 | 7.22072E−70 |
| ASB2 | 1.279981239 | 9.30747E−19 |
| CYP1B1 | 1.139473622 | 2.62417E−22 |
| KRT23 | 1.123871941 | 5.70973E−15 |
| NPPB | 1.112455481 | 6.88756E−18 |
| ADAMTS5 | 1.092258659 | 3.42678E−38 |
| Downregulated Genes: | | |
| TMEM2 | −0.380126642 | 2.75238E−09 |
| KIAA0247 | −0.41378386 | 7.76616E−09 |
| RERE | −0.415844685 | 7.37465E−09 |
| PDXK | −0.436642769 | 2.48101E−11 |
| TAP1 | −0.437523021 | 1.26613E−10 |
| CARHSP1 | −0.46243106 | 5.04133E−09 |
| NEDD9 | −0.478848357 | 6.33913E−09 |
| TAPSAR1 | −0.481961432 | 3.61579E−10 |
| CPD | −0.488085215 | 3.70771E−13 |
| CNDP2 | −0.492242807 | 2.28694E−10 |

Promoter Prediction

The genetic sequence of the region upstream of the IL-1β and BMF genes, containing the promoter sequence and potential additional regulatory sequence, was obtained from the Ensembl database. An online software (Proscan programme v 1.7) was used to confirm the chosen sequence has the featured elements of a promoter like TATA box, transcription factor binding sites etc. As a control stable podocyte cell line without any promoter (blank vector) was included in the standardization of the assay to check the authenticity of promoter sequence. Extensive PCR optimization, cloning, digestion and sequencing was then performed to optimize the promote sequence for use in the assay.

PCR and Cloning of Promotor Region

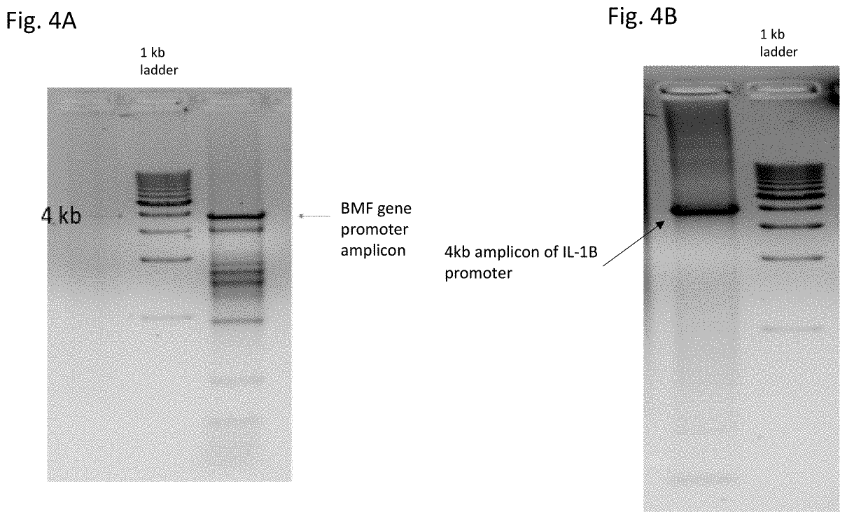

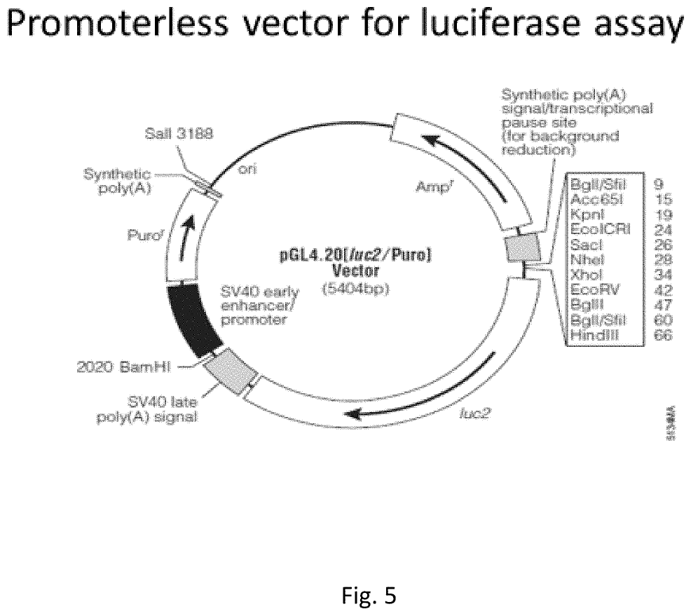

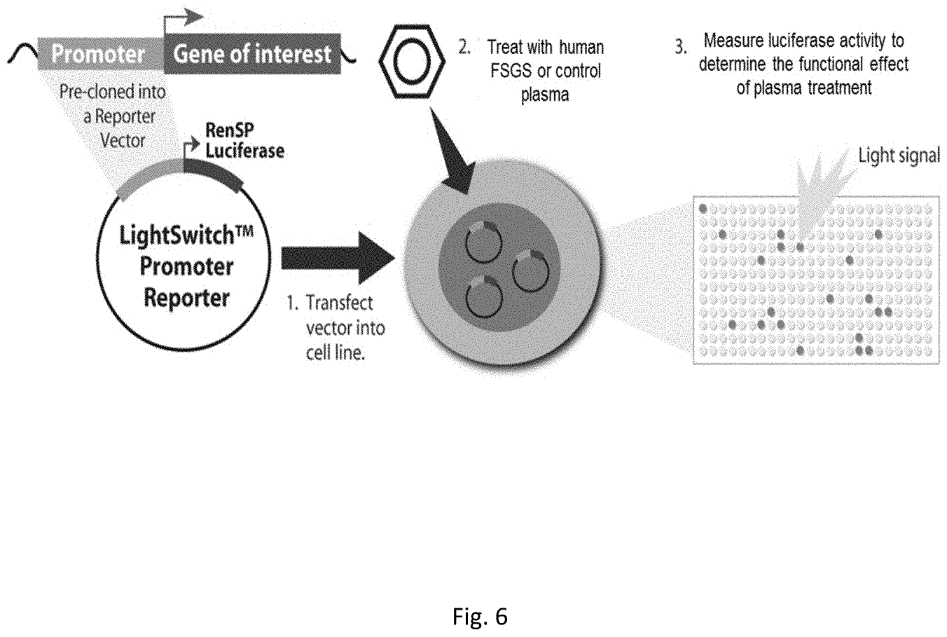

A 4 kb region of the 5′UTR of IL-1β, BMF(FIG. 4A) or (FIG. 4B) was PCR amplified and cloned upstream of a Luciferase reporter gene ORF: Luciferase reporter vector pGL4.20 (luc2/Puro) (FIG. 5 and FIG. 6). The IL-1β promoter sequence was cloned using NheI/BglII restriction sites. The BMF promoter sequence was cloned using NheI/HindIII restriction sites. The primers used are provided in Table 3.

TABLE 3
List of oligonucleotides used to amplify promoter
regions of BMF, IL1β, IGFBP3 and LAMP3 genes.

| SEQ | | |
|---|---|---|
| ID | | |
| NO: | Primer | Sequence, 5′-3′ |
| 1 | BMF | GCGGCGGCTAGCAACTATCCTTTGCAGACAGACGG |
| | Forward | |
| 2 | BMF | GCGGCGAAGCTTAAAATACGCCTGCTCGGGGCATCC |
| | Reverse | |
| 3 | IL1β | GCGGCGGCTAGCGACTGCTTTTCTGAAATGATGCAA |
| | Forward | GG |
| 4 | IL1β | GCGGCGAGATCTGGAAGCTTCTTAGGGGAGGGGAC |
| | Reverse | |
| 5 | IGFBP3 | GCGGCGGCTAGCGTCTATTGGTGGTCAGTAAGACTG |
| | Forward | ATAGC |
| 6 | IGFBP3 | GCGGCGAAGCTTGACGCCTGCAACCGGGGCACGCT |
| | Reverse | GCTTGG |
| 7 | LAMP3 | GCGGCGGCTAGCGATCAACCTCCAGCCAAACTTATC |
| | Forward | AGG |
| 8 | LAMP3 | GCGGCGGATATCAACGAAACCACCTGCTTATGAGA |
| | Reverse | AGCAGC |

Construction of the Reporter-Based Podocyte Cell Lines:

The podocyte cell lines were constructed using a standard method of DNA transfection in cells using the transfection reagent lipofectamine. Briefly, the podocyte cells were plated at a 90% confluency in a petri dish. In a sterile eppendorf tube 500 μl of Optimem Media was added followed by 24 μg of reporter plasmid DNA and incubated at room temperature for 5 minutes. In a separate tube 60 μl of Lipofectamine 2000 in 500 μl Optimem Media was added. After a 5 minute incubation at room temperature, the two tubes were mixed and further incubated for 20 minutes. 1 ml of plasmid-Lipofectamine complexes were then added drop by drop to the cells in petri dish. Cells were later selected by treating with puromycin to generate stable cell lines. These cell lines were then used in the assay experiments.

Luciferase Assay

Stable cell lines expressing luciferase constructs were tested for bioluminescence response upon treatment with the plasma of study subjects. Briefly, the cells were cultured in 24 well plates to achieve 90% confluency. The cells were washed with serum free medium, starved overnight in serum free RPMI medium and treated with patient plasma (4% patient or control plasma) diluted in 500 μl of serum free RPMI without phenol red indicator (to avoid interference in luminescence readings). Cells were incubated further for 12-16 hours. Then luciferase assay was performed using the “ONE-Glo™ EX Luciferase Assay System” (Promega # E8130) as per the manufacturer's instructions. Briefly, cells were washed and 150 μl of 1× passive lysis buffer (Promega # E194A) was added to each well and incubated further for 15 minutes at 4° C. with mild shaking. The entire lysate was collected in microtubes and spun at 5000 rpm for 10 minutes at 4° C. 80 μl of supernatant was dispensed in the 96 well white opaque tissue culture plate. 80 μl/well of One Glow reagent (Promega) was added and incubated for 2-3 mins at RT before recording luminescence in a luminometer (Berthold Centro XS3 LB960—DLR Ready—Berthold Technologies, Germany) at an integration time of 1 second.

Quantitative Real-Time PCR

To evaluate the expression of BMF, IL1β and IGFBP3 genes, the cultured human podocyte cells were incubated with plasma from rFSGS and FSGS patients. Quantitative real-time PCR (qPCR) was performed using CFX96 real time thermal cycler (Biorad) and the gene-specific primers designed using Generunner (Table 4). Three independent RNA samples were used for analysis. The gene encoding ACTIN protein was used for calibration. Data analyses were performed using threshold cycle (ΔΔC_{T}) method described by Livak and Schmittgen (Livak, K. J. et al., 2001, Methods, 25(4):402-408).

TABLE 4
List of oligonucleotides used for RT PCR.

| SEQ ID | | |
|---|---|---|
| NO: | Primer | Sequence (5′-3′) |
| 9 | IL1β-F | AATCTGTACCTGTCCTGCGTGTT |
| 10 | IL1β-R | TGGGTAATTTTTGGGATCTACACTCT |
| 11 | BMF-F | ATGGAGCCATCTCAGTGTGTG |
| 12 | BMF-R | CCCCGTTCCTGTTCTCTTCT |
| 13 | IGFBP3-F | GTCCAAGCGGGAGACAGAATAT |
| 14 | IGFBP3-R | CCTGGGACTCAGCACATTGA |

Statistical Analyses

The data were collected for experiments examining fold change in the expression of BMF, IL1β and IGFBP3 relative to healthy controls in patients with rFSGS, FSGS, MGN, and controls. The luminescence readings were normalized with control readings and relative light units were calculated and plotted as shown in the graphs. Descriptive statistics were calculated for patient samples including the proportion of rFSGS, FSGS, MGN and control patients and the mean fold-change relative to control in IL1β, BMF, IGFBP3 and LAMP3 for each group. The primary goal of this analyses was to determine if there are significant differences in fold-change in luminescence (relative to controls) of IL1β, BMF, IGFBP3 or LAMP3 between rFSGS patients and patients with either FSGS or other nephropathies like MGN. A linear mixed model approach was used to estimate mean fold change relative to controls in samples from rFSGS and patients without recurrent FSGS. The model included a fixed effect for disease type and random subject and batch effects to account for measurements collected on the same subject and samples evaluated in the same experiment. Pairwise comparisons between rFSGS, FSGS, nephropathies patients (like MGN) and controls were conducted to estimate differences in gene expression between the different groups using the Tukey honestly significant difference (HSD) adjustment to control for multiple comparisons. Generalized linear mixed regression models (GLMMs) were also fit to compare the ability of each gene to discriminate between rFSGS versus other nephropathies or control and between rFSGS and FSGS. Area under the receiver operating characteristics curve (AUC), sensitivity, and specificity with 95% confidence interval for each gene were estimated from the GLMM models. All analyses were conducted in SAS v. 9.4 (SAS Institute, Cary N.C.).

Human Subjects

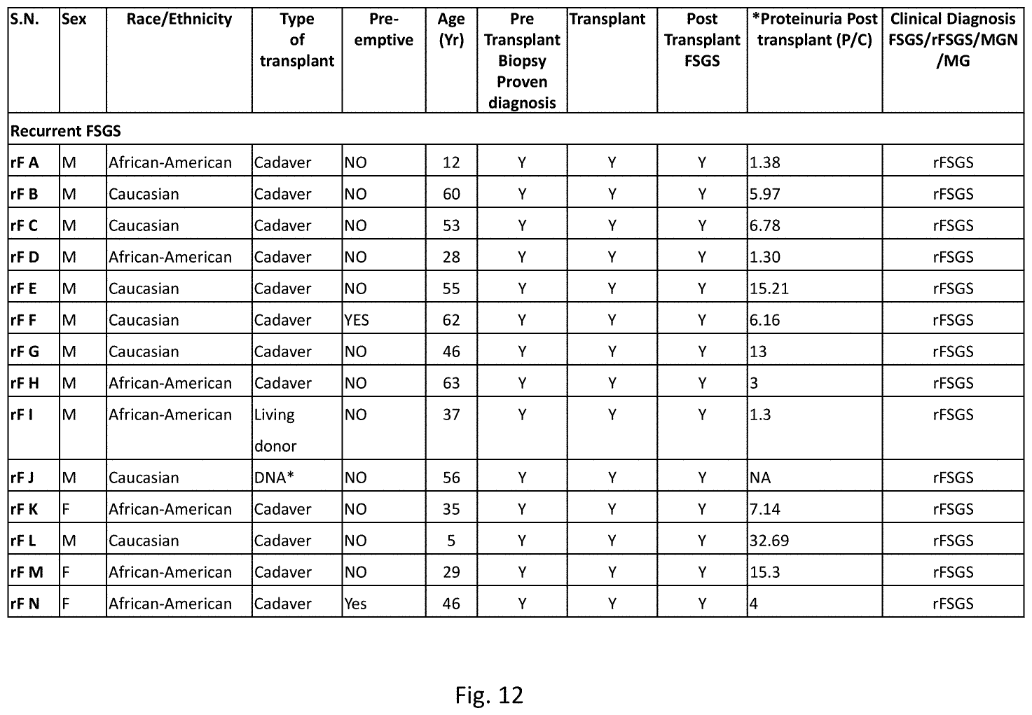

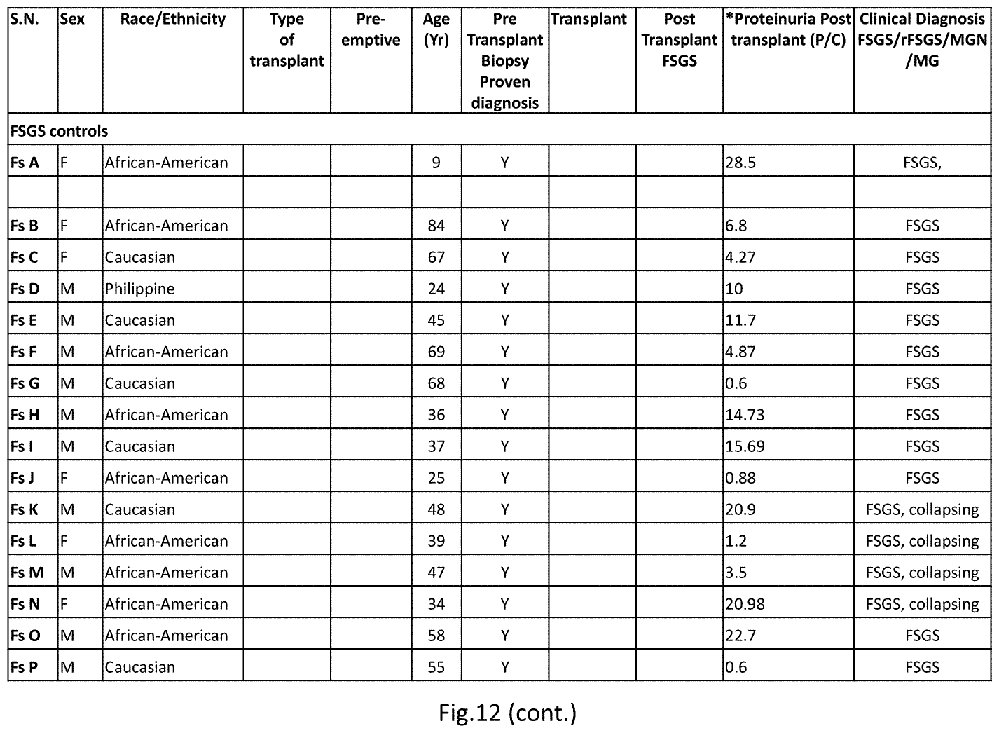

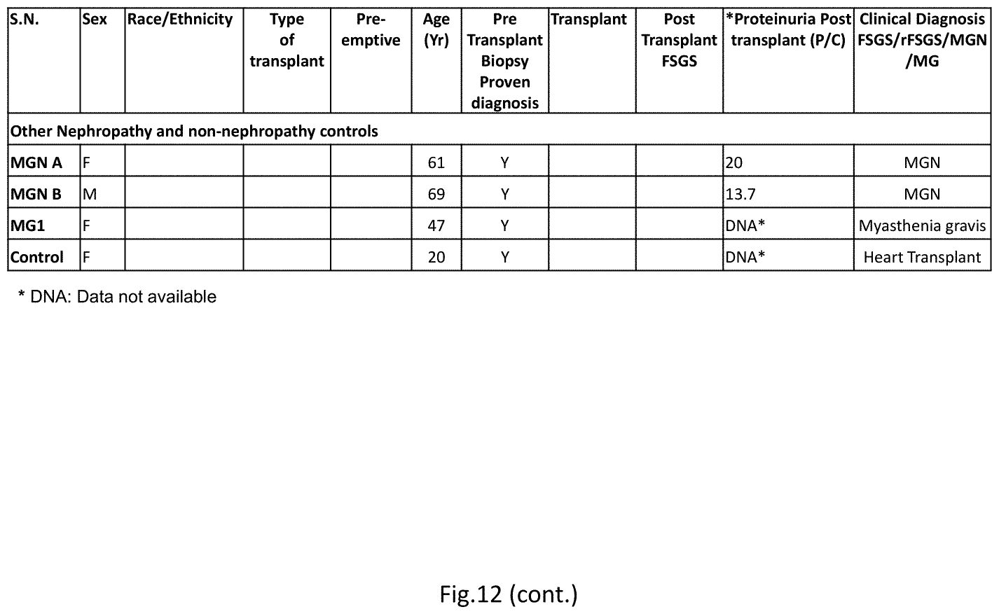

Details of the study subjects are provided in FIG. 12. Collection of blood samples were performed according to the IRB guidelines. Over the past two decades plasma primarily from FSGS patients has been collected and cataloged from FSGS patients. A very stringent criterion for selecting all the patients and controls was used for this study. The patient designated of having rFSGS had to have confirmed diagnosis of FSGS in native kidneys, undergo kidney transplants and develop nephrotic range proteinuria and/or histologic findings of podocyte effacement or FSGS within few hours to days or weeks of the transplant (Table 5). The plasma from all the listed patients were collected immediately prior to plasmapheresis, which was initiated as soon as they were diagnosed with heavy proteinuria. 30 patients were initially identified with FSGS; 14 of these patients who received renal transplant showed recurrence of FSGS within hours to weeks. Control subjects included heart transplant recipients (to account for possible nonspecific effects of immunosuppressive drugs), non-recurrent FSGS patients and patient with histologic diagnosis of membranous nephropathy (to account for nonspecific effects of nephrotic state). Plasma samples obtained from the study subjects were aliquoted and stored at −80° C.

TABLE 5
Period of recurrence of FSGS in patients (rF A-N) following renal
transplant. The period denotes when the clinical diagnosis was made.

| | Recurrence of FSGS | |
|---|---|---|
| Patients | (Biopsy) | |
| Patient A | 12 | hours |
| Patient B | 12 | hours |
| Patient C | 70 | days |
| Re-transplant for Patient C | 2 | years |
| Patient D | 12 | hours |
| Patient E | 1 | month |
| Patient F | 1 | month |
| Patient G | 4 | months |
| Patient H | 4 | months |
| Patient I | 2 | years* |
| Patient J | 2 | years* |
| Patient K | 2 | years |
| Patient L | 5 | days |
| Patient M | 9 | years |
| Patient N | 10 | years |

*patients redeveloped FSGS, but the official reporting was delayed.

The results of this example are now described.

Development of a Cell-Based Assay for rFSGS

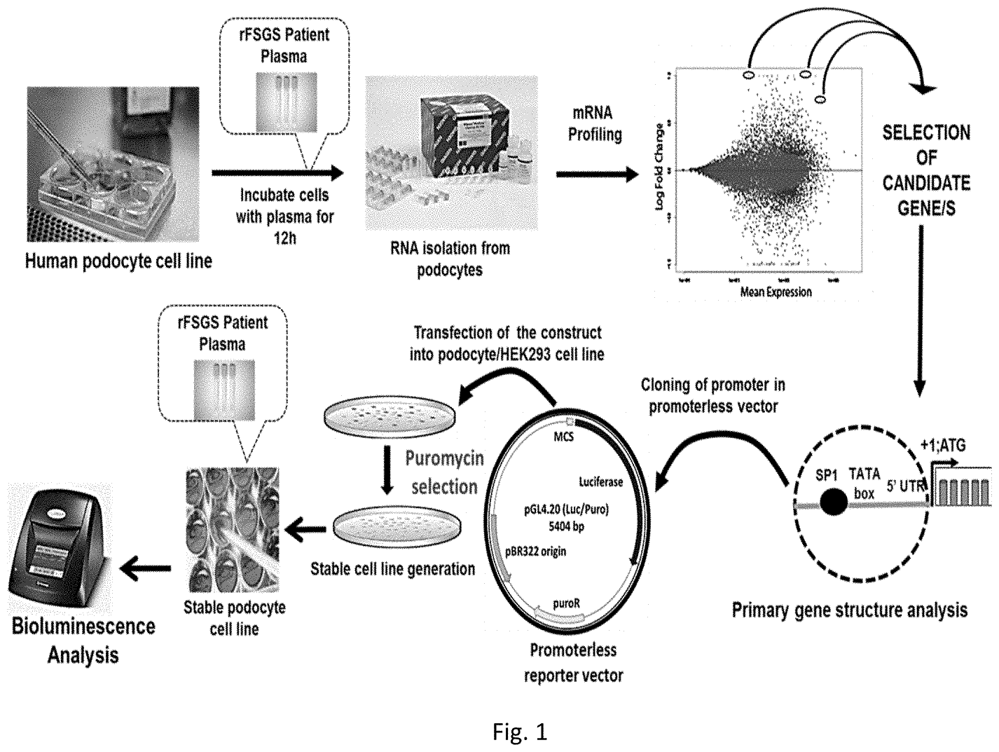

Without being bound by theory, since FSGS primarily targets podocytes, and the plasma from FSGS patients has been shown to induce podocyte damage (Harris, J. J. et al., 2013, J Pathol., 229(5):660-671; Kachurina, N. et al., 2016, Am J Renal Physiol., 310(10):F1148-1156), it was hypothesized that rFSGS patients have unique component/s in their plasma, which when added to cultured human podocytes can induce a specific set of genes and pathways involved in cellular damage including apoptosis. It was further hypothesized that identification of these rFSGS responsive genes can be performed using the mRNA profiling of rFSGS plasma treated podocytes. Promoter sequences from these genes can then be used to construct reporter-based podocyte cell lines in which, the promoter activity can be selectively induced by treating with plasma from rFSGS patients (FIG. 1). Furthermore, using these cell lines an assay can be developed that serves as a diagnostic tool for detecting rFSGS. A schematic representation of this concept is presented in FIG. 1.

The podocyte cell lines were tested for their response against plasma from various rFSGS and other nephropathy patients. 4% final concentration of plasma was used for induction. The assay was performed as described above.

The Plasma from rFSGS Patients Induces Podocyte Actin Cytoskeleton Damage

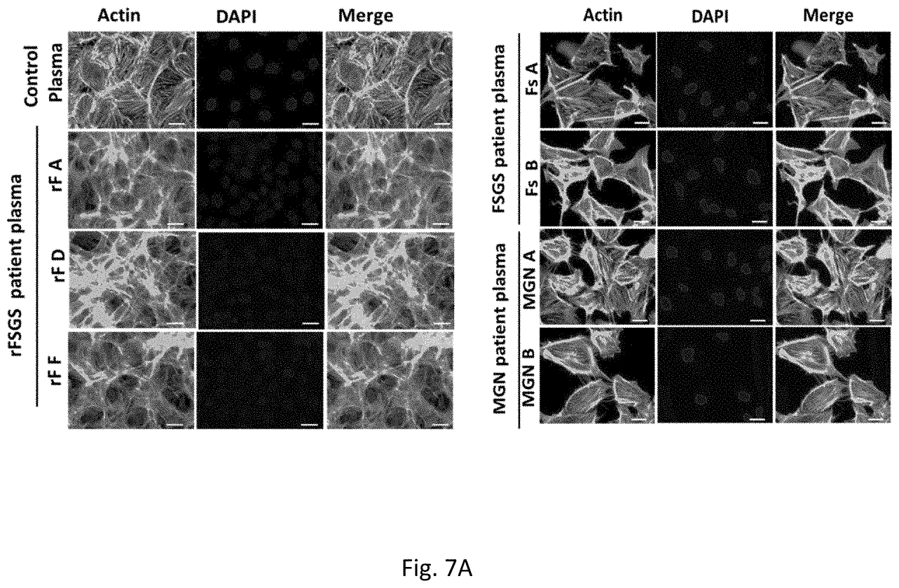

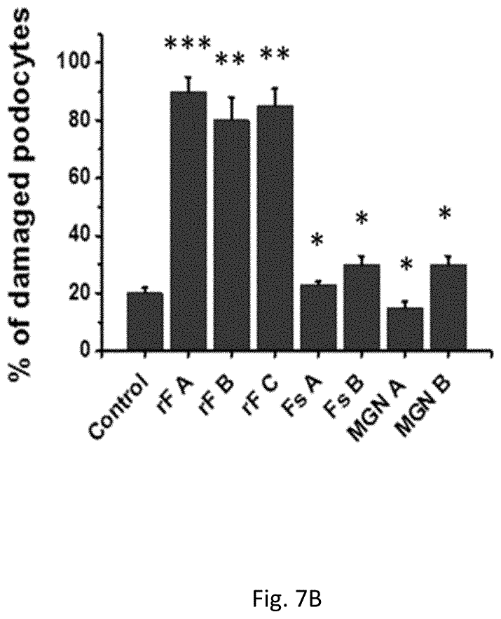

Since plasma from rFSGS patients has been shown to induce podocyte actin cytoskeleton damage leading to podocyte loss (Coward, R. J. et al., 2005, J Am Soc Nephrol., 16(3):629-637), it was first tested if the plasma from rFSGS patients to be used in this study induces podocyte cytoskeletal disorganization. Thus, cultured human podocytes were treated with plasma from three rFSGS patients (rF A, D and F), two non-recurrent FSGS patients (Fs A and B) and two membranous nephropathy (MGN) patients (MGN A and B). As expected, the immunofluorescence analyses revealed significant changes to the cytoplasmic distribution of actin stress fibers in rFSGS plasma when compared to the cells treated with non-recurrent FSGS, MGN and control plasma, which showed predominantly cortical distribution (FIG. 7A). Quantification of changes in the actin cytoskeleton staining patterns showed >80% cells with altered actin cytoskeleton morphology depicting cellular damage in rFSGS plasma treated podocytes, whereas the podocytes treated with non-recurrent FSGS and MGN plasma showed <30% damage and the control plasma showed <20% cellular damage (FIG. 7B). These results are consistent with previously published observations, where similar changes to podocyte actin cytoskeleton by rFSGS plasma demonstrating its potency have been reported (Coward, R. J. et al., 2005, J Am Soc Nephrol., 16(3):629-637; Fornoni, A. et al., 2011, Sci Transl Med., 3(85):p. 85ra46). It should be noted that the control plasma was derived from a heart transplant patient, who had a similar drug profile as the rFSGS patients with no history of nephrotic disease.

Plasma from rFSGS Patients Induces Differentia Expression of Several Genes in Podocytes

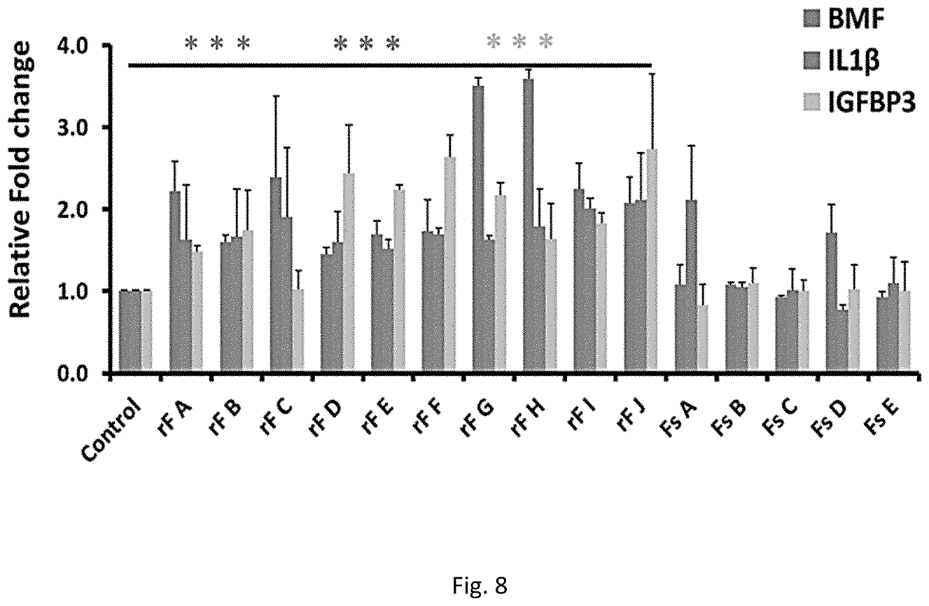

Since all forms of FSGS primarily target podocytes (Harris, J. J. et al., 2013, J Pathol., 229(5):660-671; Kachurina, N. et al., 2016, Am J Renal Physiol., 310(10):F1148-1156), which ultimately leads to podocyte dysfunction and death, it was next evaluated if treatment of human podocytes with rFSGS patients plasma induces genes involved with cellular damage/apoptosis. Indeed, 12 hour treatment of cultured podocytes with plasma from two different rFSGS patients (rF A and D) showed differential expression (DE) of several genes (supplementary info). Although several genes were differentially expressed, BMF (BC12 modifying factor), IL-1β (Interleukin 1 beta), IGFBP3 (Insulin growth factor binding protein 3) genes were selected for further validation. These genes were upregulated and have proapoptotic functions (Lee, H. S. et al., 2014, Arthritis Rheumatol, 66(4): 863-873; Martin D. S. et al., 2002, J Biol Chem., 277(37):34239-34246; Puthalakath, H. et al., 2001, 293(5536):1829-1832). To further validate the upregulation of these three candidate genes, quantitative RT-PCR was performed using gene specific primers. Expectedly, podocytes treated with rFSGS patient plasma showed significantly increased expression of BMF, IL1β and IGFBP3 genes, when compared to the non-recurrent FSGS and control patient plasma (FIG. 8). Additionally, LAMP3 (Lysosomal-Associated Membrane Protein 3) gene was selected as a negative control, which was not upregulated in this analyses. Next, promoter regions from all the four genes were identified (based on, the published literature (Shirakawa, F. et al., 1993, Mol Cell Biol., 13(3):1332-1344; Zhang, Y. et al., 2006, Cell Death Differ., 13(1):129-140) and selecting a region 2.5 to 4 kb upstream of transcription start site), PCR amplified (using primer sets listed in Table 4), and cloned into a promoterless vector (Promega (pGL4.20[luc2/puro])). The final constructs were verified through sequencing and transfected into cultured human podocytes and stable cell lines for each promoter construct were created using puromycin selection.

The Cell Lines Containing Firefly Reporter Constructs Selectively Responded to rFSGS Patient Plasma

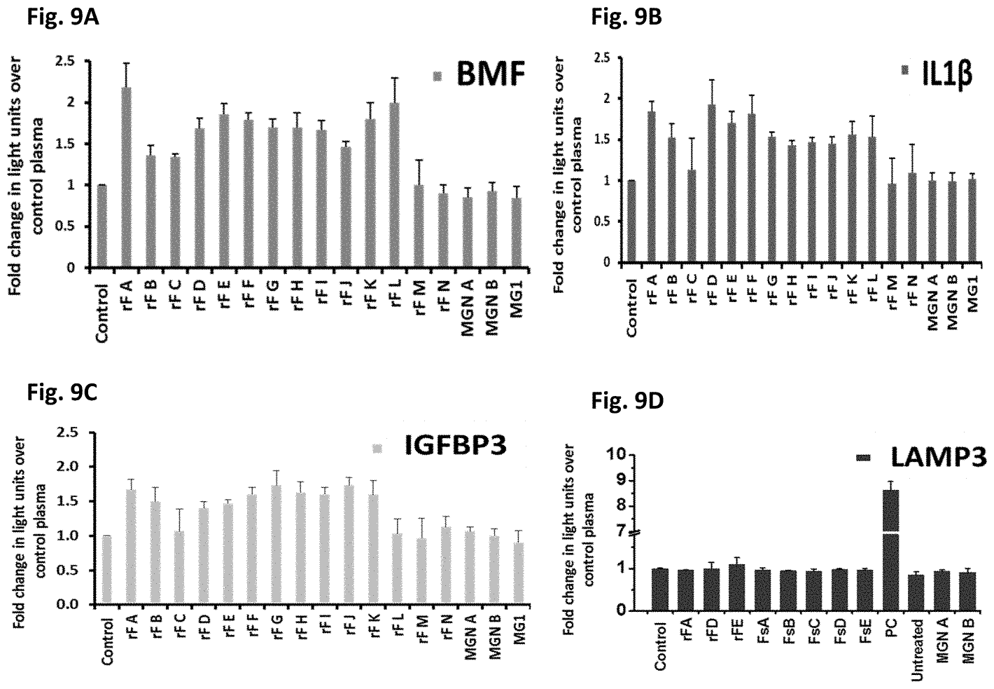

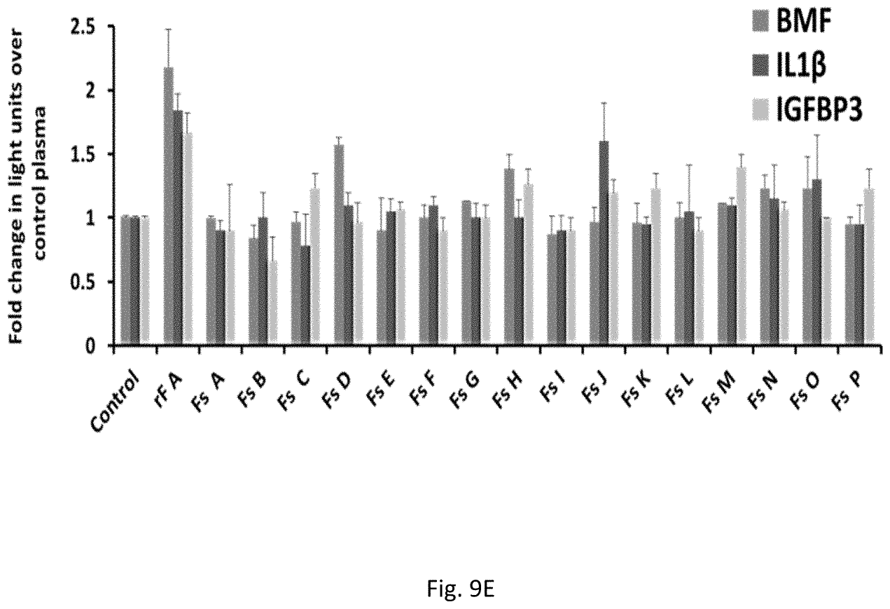

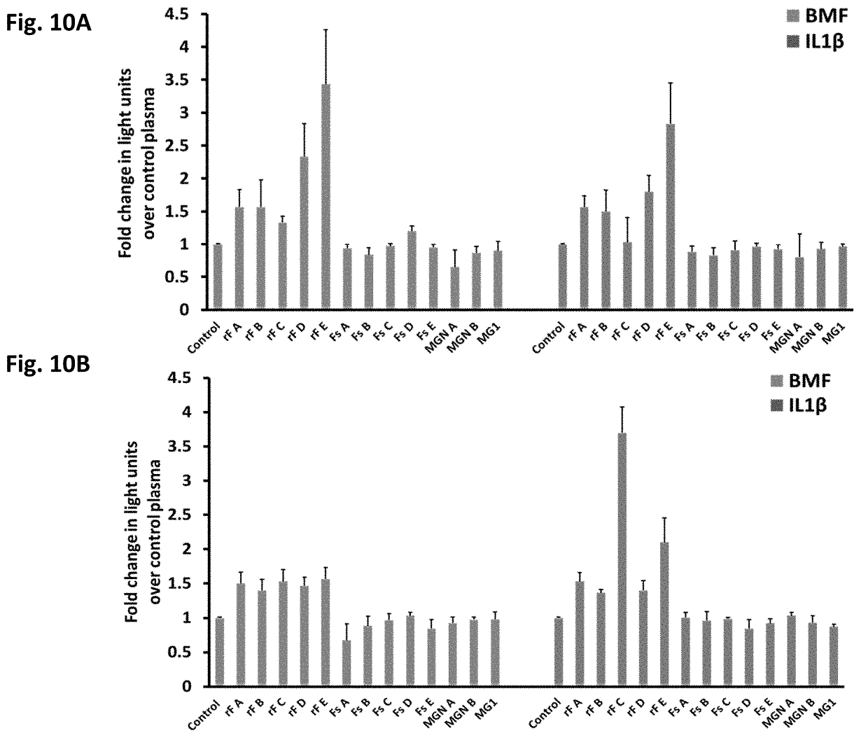

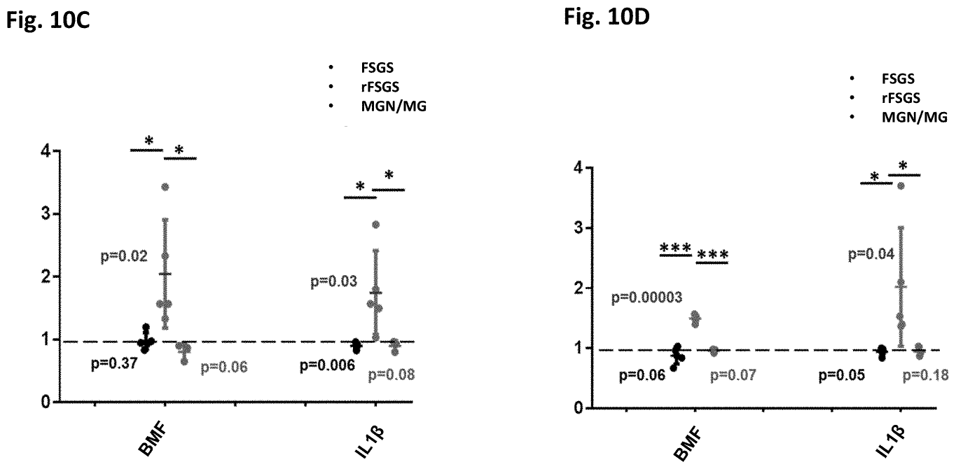

To determine whether the promoter driven reporter in these cell lines are activated by rFSGS plasma, reporter activity of these cell lines were measured following treatment with plasma from various rFSGS patients. In addition to the plasma from two rFSGS patients (rF A & D) that were used for mRNA profiling, plasma from 12 naïve rFSGS patients (rF B, C and E-N), were used. Strict criteria were used to clinically confirm the diagnosis of FSGS recurrence in all these patients (FIG. 12 and Table 5). Remarkably, all the three cell lines (BMF, IL1β, and IGFBP3) showed significant increase in luciferase activity over the control plasma when plasma from the rFSGS patients was added to these cells (FIG. 9A-FIG. 9C). Additionally, although the response was variable for each patient (rF A-N), majority of rFSGS patients showed an increase in luciferase activity over the control in these cell lines (FIG. 9A-FIG. 9C). To further determine if the cellular response is independent of host cell line and specific to the reporter gene promoter, the reporter constructs were also transfected into HEK-293 and COS7 cells, and stable cell lines were created and tested in the same assay. Most notably, both the cell lines positively identified rFSGS patients and did not respond to plasma from either a control or non-glomerular disease patient (FIG. 10A-FIG. 10B). In contrast, the control LAMP3 podocyte reporter cell line, when treated with plasma from rFSGS, non-recurrent FSGS and other disease patients, did not show any induction of the reporter activity further confirming BMF, IL1β and IGFBP3 as genes responsive to rFSGS patient plasma (FIG. 9D). To further validate the LAMP3 reporter cell line, the induction of LAMP3 promoter with CD63 antibody was used as a positive control (FIG. 9D).

To further establish the specificity of the approach, the response of these cell lines towards plasma from other glomerular disease patients including non-recurrent FSGS (Fs) and Membranous glomerulopathy (MGN) was tested. Unlike the rFSGS plasma, the plasma from these patients did not generate a consistent response with all the cell lines tested (FIG. 9E).

The Cell-Based Assay Detects rFSGS Patients with High Sensitivity and Specificity

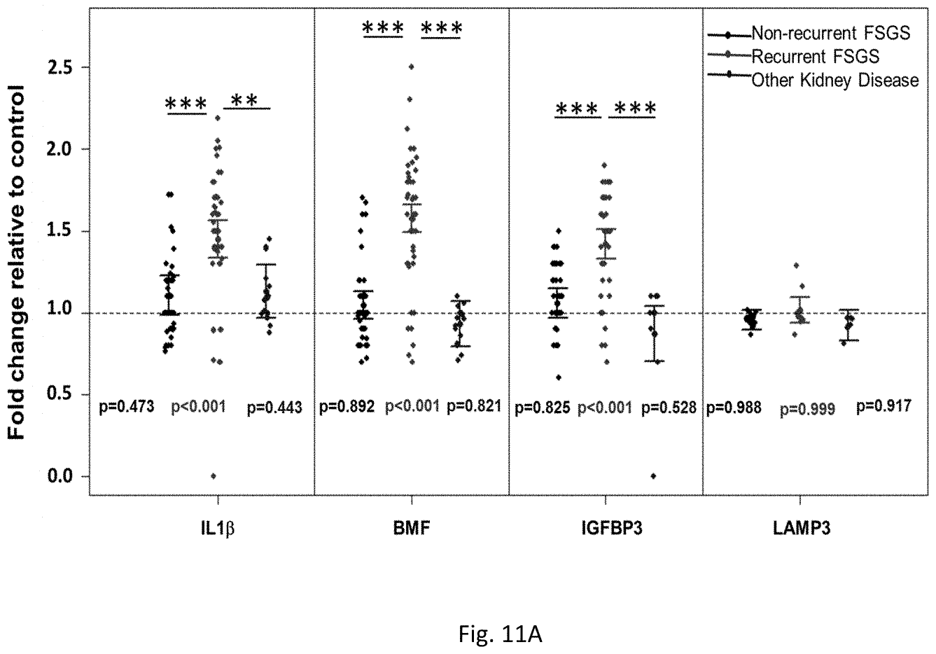

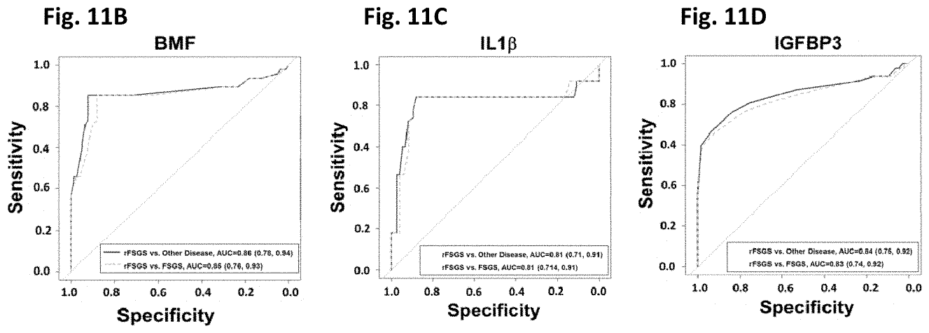

To evaluate the specificity of this assay, differences in mean fold-change response from BMF, IL1β and IGFBP3 cell lines between rFSGS patient samples and other nephropathies were evaluated. Table 6 shows the mean fold change relative to controls in IL1β, BMF, IGFBP3, and LAMP3 for rFSGS patient samples, non-recurrent FSGS samples, MGN and control samples (Myasthenia gravis (MG) and heart transplant.) The mean fold change in reporter activity with standard error for all promoters is presented in FIG. 11A. The initial comparison of relative fold-change in expression showed that relative to controls, there was a significant increase in the expression of BMF, IL1β and IGFBP3 (p<0.001 for all) in rFSGS samples (FIG. 11A). In contrast, no significant change in the expression of LAMP3 reporter relative to controls in rFSGS samples was noted (p=0.999). Additionally, there was no significant difference between samples from patients with non-recurrent FSGS or other nephropathies relative to controls (FIG. 11A). Further comparison of rFSGS with all the other samples showed a higher fold change for BMF, IL1β and IGFBP3 gene promoters in the rFSGS group (p<0.001); whereas no differences were noted for LAMP3 gene promoter activity (p=0.398). Individual comparisons of IL1β or BMF between groups showed that rFSGS samples exhibited higher relative reporter activity when compared to MGN/controls or non-recurrent FSGS (p<0.001); whereas IGFBP3 promoter activity was different at the level of p<0.001 for non-recurrent FSGS and p=0.015 for MGN/controls.

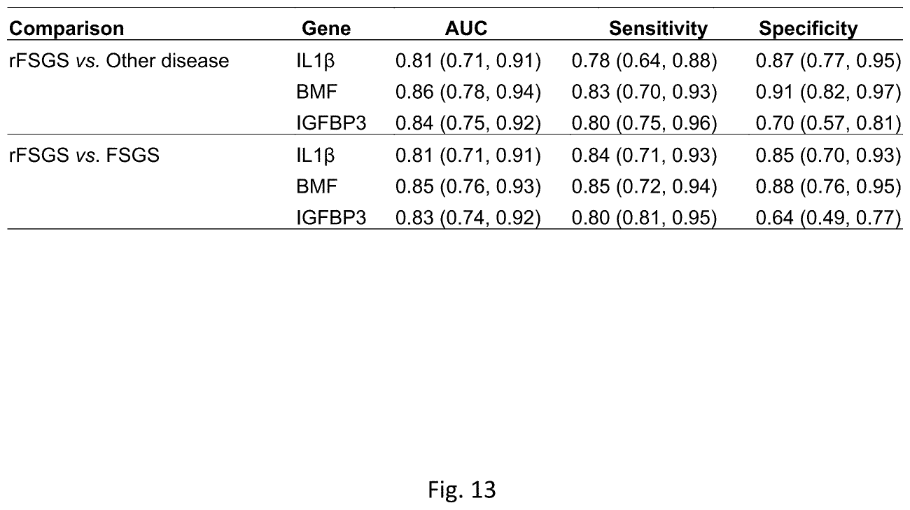

To further evaluate the discriminative performance for each promoter, the area under the receiver operating characteristics curves (AUCs) for models discriminating between rFSGS versus nephropathies and rFSGS versus non-recurrent FSGS based on fold-change values for IL1β, BMF, and IGFBP3 promoter cell lines were estimated. The AUCs for these models ranged from 0.81 to 0.86 (FIG. 11B-FIG. 11D). Additionally, the estimated sensitivities across all models were greater than 80%. The specificities for IL1β and BMF were greater than 85% for rFSGS vs. all other nephropathies and for rFSGS vs. non-recurrent FSGS. However, the specificities for IGBFP3 were slightly lower at 70% for rFSGS vs. all other nephropathies and 64% for rFSGS vs. non-recurrent FSGS. Discriminative characteristics with 95% confidence intervals for all genes comparing rFSGS vs. all other nephropathies and rFSGS vs. non-recurrent FSGS are shown in FIG. 13.

TABLE 6
Mean fold-change in the expression relative to controls by disease
status. Values are reported as mean (SE) and are estimated from the
linear mixed model.

| Gene | MGN, MG and | FSGS | rFSGS |
|---|---|---|---|
| Promoter | Control (n = 4) | (n = 16) | (n = 14) |
| IL1β | 1.13 (0.082) | 1.11 (0.061) | 1.45 (0.058) |
| BMF | 0.93 (0.071) | 1.05 (0.043) | 1.58 (0.043) |
| IGFBP3 | 0.87 (0.085) | 1.06 (0.046) | 1.42 (0.046) |
| LAMP3* | 0.92 (0.047) | 0.96 (0.031) | 1.02 (0.039) |

*Mean FC values for LAMP3 were calculated based on 3 other disease, 5 FSGS, and 3 rFSGS samples.

### Example 2: Generation of Stable Reporter Cell Lines

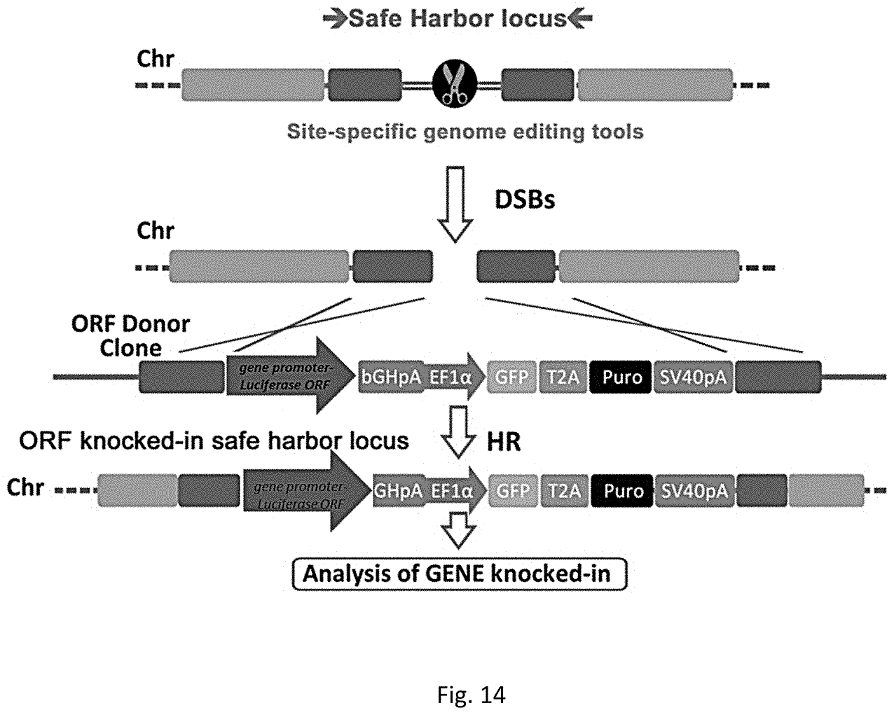

To avoid the variability in transgene expression in cells and random insertion of the transgenes in podocyte genome, and to achieve a robust response, a CRISPR/Cas9-based gene editing system is employed. One copy of the luciferase reporter gene was inserted in a CRISPR targeting construct. The strategy to generate a CRISPR targeting construct is shown in FIG. 14 and is outlined below. First, the Genome-CRISP Cas9 human AAVS1 Safe Harbor knockin kit-Puro (Cat # SH012) from GeneCopoeia, Inc. is used to construct targeting CRISPR donor constructs for the promoter region of the BMF candidate gene. The final donor construct contains the 2.4 kb BMF promoter region followed by one copy of the Luciferase open reading frame (ORF). The targeting constructs are confirmed by DNA sequencing, transfected in podocytes and after few cycles of puromycin selection several individual clones were screened for their GFP expression. These putative clones were then subjected to PCR analyses for the identification of correct integration of donor cassette at AAVS1 site using outside and inside primers. These cell lines are then tested for a positive response towards rFSGS plasma, using the existing sample database used in Example 1. These cells are plated in a 96 well format and treated with rFSGS plasma as described in Example 1 (FIG. 6). Treated cells are processed for luciferase assay using a luciferase assay kit (ONE-Glo™ EX Luciferase Assay System from Promega, Cat. No: E8130) and following the manufacturer's protocol. The luciferase activity is measured using a luminometer system. A positive response from the plasma of a patient with definitive diagnosis of rFSGS based on earlier stated criteria from renal biopsy and FSGS recurrence, is used as a reference control. FSGS and non-FSGS patient plasma samples are tested to determine if they generate a similar or differential response in this assay.

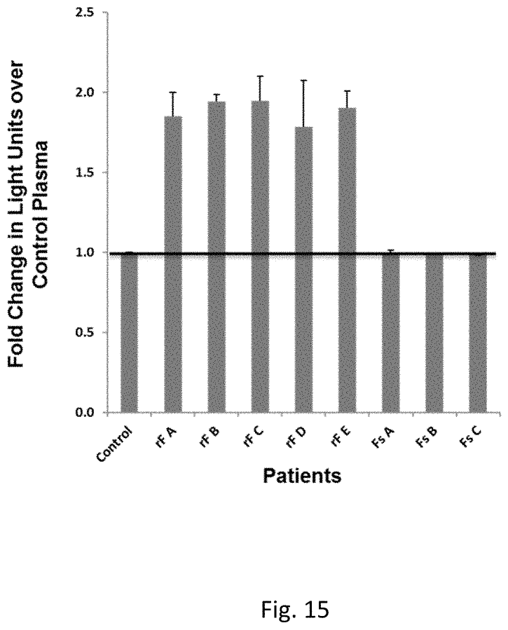

The Hek293 cell line constructed using CRSPR/Cas9 mediated knockin of the BMF promoter-reporter construct also selectively responded to rFSGS patient plasma: To reduce cellular variability, the ‘BMF promoter-reporter’ cassette was knocked in to the HEK293 cell line at a genomic locus (AAVS1 site) using Crispr Cas9 technology. The cells were treated with patient plasma [rFSGS (rF A-E) and non rFSGS (Fs A-C)] as indicated and the reporter activity was measured. Remarkably, this cell line showed significant increase in luciferase activity (over the control plasma), when plasma from the rFSGS patients was added to these cells and did not respond to plasma from either a control or non rFSGS disease patient (FIG. 15). The response from this cell line was consistent, less variable and slightly higher when compared to plasmid-based cell lines.

Primers used to clone the insert “BMF+Luciferase ORF” in donor vector:

BMFprom2400EcoRVF:
(SEQ ID NO: 18)
GCGGCGGATATCTAATAAGTGTTTGCACCACTGCACTGCAGCCTGG
LuciferaseXbaIR:
(SEQ ID NO: 19)
GCGGCGTCTAGATTACACGGCGATCTTGCCGCCCTTCTTGG

### Example 3: Standardization of the Cell Lines to Boost the Assay Response

The exact diagnosis of glomerular diseases rests on kidney biopsy findings, an invasive procedure associated with risk of complications and not safe or feasible to perform in a substantial number of patients. Regarding rFSGS, few reports have emerged in the past where claims have been made about the identity of humoral factor/s as the underlying cause of rFSGS, none of them have been clinically confirmed. This has made it difficult to develop a diagnostic assay for rFSGS patients, who continue to suffer from the lack of transplant success, and are relinquished to dialysis. Further, glomerular enlargement assay, antibody and cell-culture based approaches were proposed to diagnose rFSGS, but these assays are time consuming, technically challenging, and involve a panel of antibodies that make them expensive to perform and therefore are unlikely to be adopted commercially. rFSGS leads to unsuccessful kidney transplants, which lead to an enormous cost burden financially and to the resources of the healthcare system. An assay that would reliably and reproducibly predict rFSGS would be immensely beneficial in reducing the overall cost of unsuccessful renal transplants, as these patients could be treated with effective prophylactically and/or perioperative treatment, such as plasmapheresis and/or high-dose cyclosporine.

Assay Optimization by Measuring Assay Response Over Time

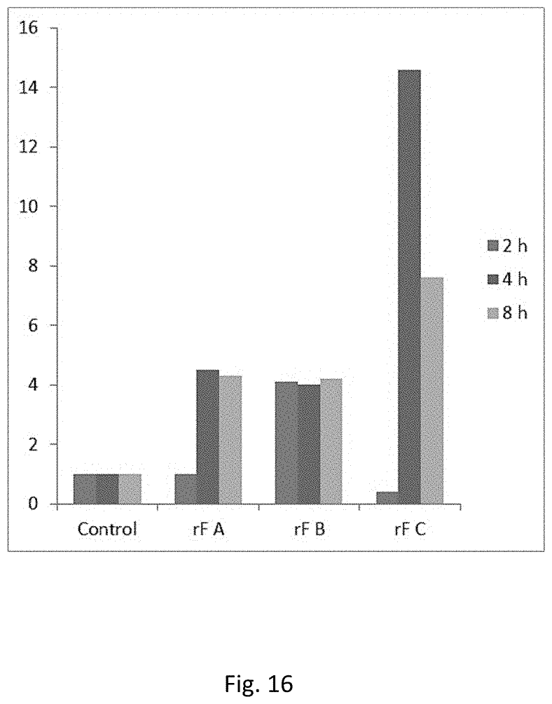

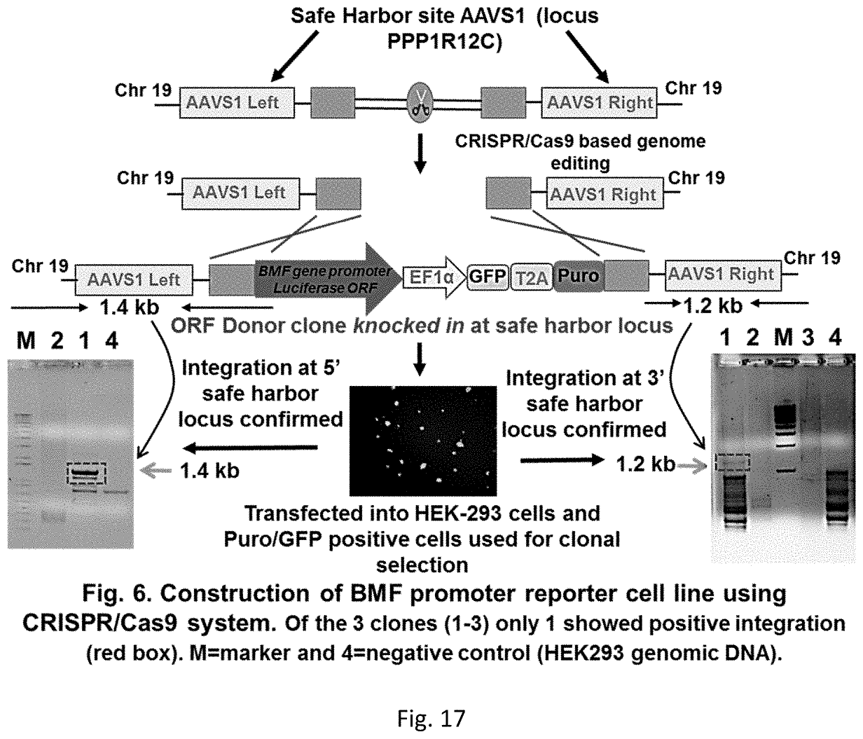

To determine optimal time period during which the assay response is maximal, a time course was performed, where the BMF cell line was incubated with patient plasma for different time intervals ranging from 0-8 hours. As shown in FIG. 16, the assay response was maximal at 4 hours where more than 12 fold induction was observed. This is a significant enhancement over the original response where only 2 fold induction could be seen (FIG. 9E). The subsequent assays will use this timing for further standardization.

Generating CRSPR-Cas-Based Constructs that were Cloned into the HEK293 Cell Line

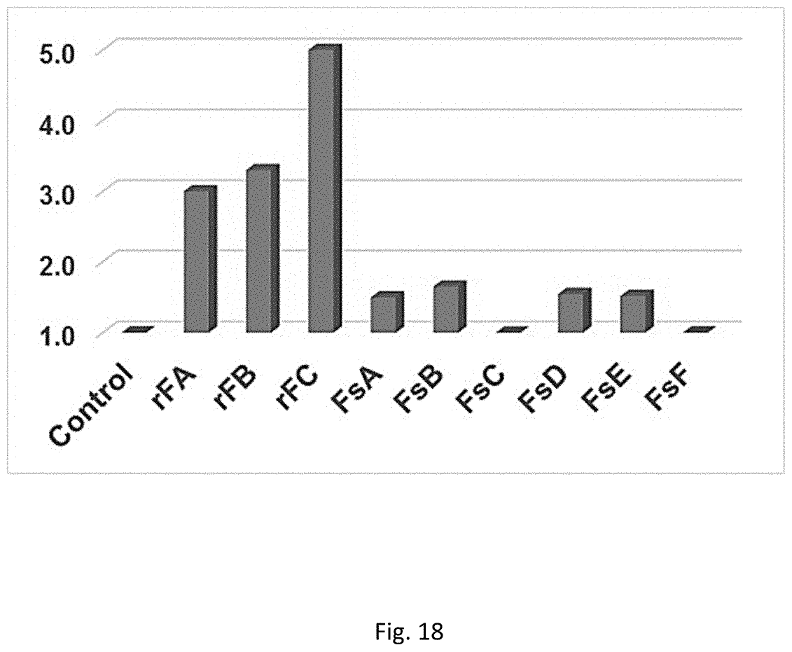

The stable cell lines used in preliminary studies demonstrated more than 80% sensitivity. However, the level of induction observed in them was modest. To generate cell lines that show maximal induction, high responsiveness and consistency in our assay, we are employing a CRSPR/Cas9 approach. Since the assay is cell line independent, due to their excellent and simple growth characteristics and superior transfection efficiency, either HEK293 (Human embryonic kidney) or Cos7 cells are used to construct our chimeric cell lines. As a primary approach, tested whether a directed insertion of candidate gene promoter-reporter sequences at the Safe Harbor site AAVS1 (locus PPP1R12C) in HEK293 cells using a standard CRSPR/Cas9 system will generate a cell line that shows uniform expression of the transgene and therefore, higher consistency and lower variability. To demonstrate the ability to utilize this system in generating the promoter-reporter cell lines, the shortened BMF promoter was used, based on published results, and the 2.4 kb promoter with luciferase reporter sequence was cloned in the Crispr/Cas9 vector from GeneCopoeia Inc (FIG. 17). The construct was sequence-verified, transfected into HEK293 cells, the clonal selection was performed (GFP positive cells, FIG. 17) and the clones were tested for site integration. A clone with successful integration at 5′ and 3′ ends was verified through PCR (FIG. 17) and tested in the assay to determine its ability to respond to patient plasma. The results presented in FIG. 18 show that the response from this cell line ranged from 3-5 fold, which is significantly higher than the original response of only 1.5 fold (FIG. 9E).

A similar approach is used for the development of three other cell lines from IL1β, IGFBP3 and Lamp3 (control) promoters, where the promoter region (˜2 kb is identified) followed by the Luciferase open reading frame are cloned in the Crispr/Cas9 vector and used to generate stable HEK293/Cos7 cell lines for the assay. Further validation and screening of clones are performed as per the manufacturer's protocols.

### Example 4: Promoter Sequences

SEQ ID NO: 15
BMF promoter sequence
aactatcctttgcagacagacggataacagataaagtccctttgatagggcaagtccggcaagggaaggacacccacgtct
tgcagatccctggtctttcttacatatatgagctcttctggaagtagttaagggggtcaggcaccatgtgctcccccagcaag
accccaaccttacccataaatcagtttcccatctaacccctccctgggggttgagttacaaacctcagtaaccccagggcag
cctgcctggtaattcctattattcagctgtcagctggttttttaaaaaccgccctttccagtgatccagttactggagttttagagc
catttcttgtctcagagagaaatcctatagccaggtgagacaatggagagggctcctgcccgctttctacaggagctgggttt
tctctctggttgagagcagcctccccttaactctgtatccctccaagattgtcagggaaggccctcctccctatcagcctccgt
gctgatgcctttctctggggacccttctcccagagccagcatcagttagcagaggagaaaacaaattgagacaaggtgcag
gaagagaatgagagaaaggaaattctccaaaatagaggttgctgtgactgcactgtgtatttgctccgagcccagtcaaaa
gctcagggttcatctgttcccagggtgctgctattggtcagggggctcagagcccagatctgcagcacagcaggtcctgat
actgctgagtccatgcagtgctgaaagggggaaaagggcatagagggaggagtctgctttgacagtgatgagaggtggg
ggtgaggctcctgacctccctgacccctttatccctgacagtccttctctggtccctctagctcagaaggctgggccccttgct
gcttgaccttagagactgagagcgcaggaggcaaagccacaggtgtctttcagaaccagcctgagggagcaggctgtga
agacctgggagagaggtcttaaaataaatatcaatagtgatgtgaccctccccatatcttgcaaactgccagaccccctgag
atactccctcctgttcctcagcccagttctgtcacaatgacgattttctgaaacctttatagggccacagaccactttatgaaact
gttgtaagctatggatccaccccactcacaaaaatgtttgtgtgtacccacaaacacagtttagccagtttcaggggttcataa
accctaaagctcaggactctgatgagcctgatgagtacatagatcccatatcaatatcccattcttggctgggtgcggtggct
catgcctataatcacagcactttaggaggccgaggtgggaggagtgctcgaggccaggagttcaagaccagcctgggca
acatagtgagaccctgtctgtacaaaaataaaaaatttaaaaagtagctggctgtggtggtgcacacctgcagtcccagcta
cttgggaggccgaggtgggaggatcacttgagcctgggagattgaggctgcaataagctgtgtttgcaccactgcactgca
gcctgggaaacagagcaagaccttgtctcaaaacaaacaaaaagaacccattctctcagcttctttgccttgtggacccaatt
ttaccatgcagggctgtgatgaagcacacagagctttagcatctcaggtgggtcaggagagctacgatggccccagccct
cagggcctgtgctgtcccagagggcagattcagagctgctgtggccttgagtttcactcttgaaccgaaggaatgctcagg
ggcaccactggccagcctgagacactggtctgataggaaagcaaatgagaagagaaggatggaaggaaggagcggaa
gggagtcggtgcattggaaatcacggagggctgccggtgcccttcacagaggctaggtttggtttctgtatctccctacatgt
agcaggagtgcaaggtcactctgaccagctcagacatgcacttttggccaagcctctgggtaaaataatatacctccttttgg
cttagtgccttttgctggaaatctatcaatttcgggggactgtgctactggctaaccttgactccccactctttcccttttccctctt
tgtcctttggagacagtatttttctattctagcaaccacagccctcaatatggctggttcattctgtttcaggtgacttctgaggga
gcaaaggaaagaatgatgggaaaaaggtacaaggagacttcaggctctattgggctgaaggtcccagctccactgaactt
aaatctaagtttacaacccagagagcagctcggtggtcagagcactgctgagccctactgcccctggtgggtcagggttgtt
gcaggactggctccctctcactgctctctggactcatcattttatctttcctaaaactcaggcttggtcaagtcactcatctccaa
taaaatctttgctgcctcccctttataaaatacagattcttcagtctggcattccagggacacctctgtagtctgacctgaaccca
cctgcccatgcctaccgttcgtaaatctcagtgtgcacccaatgatccagtcaccaagcctccctctgctccctgcacaggta
agctctccagctcagcacttcagtctgctacccttaggcctgggctattctttttattttattttatttttattttttgagacggagtc
tcattgtcgcccaggctggagtgcggtggcgctatctccgcccactgcaaccttcacctcctaggttcaagcaattctcctac
ctcagcctcccaagtagctgggattacaggcatgcgccaccacgcccgcctgatttttgtatttttagtagagacagggtttcc
cgtgttggccaggctggtctccaaccactgagctcaagtgaaccacccgcctcggcctcccaaagtgttgggattacaggc
gtgagacatgtgcatctggttttccttattgaaaaatatttcccattcaatttaaaagctgtctccctgaggccttctccagcctttg
tattctgaatttttctgcattcctcaatagtcattatccttggaatgaatacaattccaataacttttcactattttcagggctacctct
cctagtagactgtaagctccttgagggcagacgccaggtttctggcactcagcccaatatctggcacagagtaggtgctcg
gtaaatgcctgtgaagtagcccatagactcccttacgcggtctgcaggacatgctgctctcctggcagcaccagcacagtct
ctaaatgctgccatatgcgagatatgtgtcaaccgctcaagcagccccggccttcttgagcgctccgcttctcagccaggtg
cttattttgccagtgcccacgcccagtcaagaagaggacctaagggctcccctggatgtgtttgtttcaaacacaccttcagc
tttggagctgcagttttcttccgacctgctcggcaggcgggagggagctttcccctgaggctgggatcccatagggacccg
caccctgcaccctgcacccactggacgcaccagcctcataaaaaaactccccgcctccttcccccctccctttgtggacgc
gcagcaattattctgcccattgccgtgaaaaagaagacaaaagttactttggcgccccctctcccacacctaatacagagga
ttcagggactctctggcgcttccagagcctgtgttagggacagaatccgcactggcgacggcgctccgactgcgcttctgg
cgacggtcggaattttgctcggccccttgcaatgtttccatgggaaggttcgtacattcgtgaccgtccctggcagcggccc
agcccgggacttggcgcttcactcgccattggtcagtcctcggcgtgacgcgcaggggggcggggcctcatcagctgttt
gcgggatgccccgagcaggcgta
SEQ ID NO: 16
IL1β promoter sequence
gactgcttttctgaaatgatgcaaggttgagtagttgtacctgaaacatttattatctggccctttacagaaaatgtttccagacc
ctggataagtggtaccagagccccctctgtttgtggtcccctctcttatacccactaggtgtgagaaaagacatagagtagga
gagccctgccatccatcttacccacccaggggctttttctgatggatccaaaggaaggacaaggtcttattggtctcccagaa
ctgacataacaactccgacatcagggaaaagccattggagactacatagctcgccagccccagccacctgctcatatatct
aagccctccttgttctagaccagggaggagaatggaatgtcccttggactctgcatgtccccaatctgagaacctggatcca
agagggagaagaagcccattggagatgatgccataaaggaagtggaagcgatatgataaaaatcatagtgcccattccca
aataatcccagaagcagaagggaaaggagagaaatatccacaaagacaggtgtgggtacacacaacatttttcatacttta
agatcccagagggactcatggaaatgatacaagaaaatgactcataagaacaaatattaggaagccagtgccaagaatga
gatgggaaattggggaaaatgttgggggcagattgcttagttctgttctaagcaagagggtgaacaaggaaggaacagctc
actacaaagaacagacatcactgcatgtacacacaataatataagaactaacccatgattattttgcttgtcttcttgttcaaaat
gattgaagaccaatgagatgagatcaaccttgataactggctgcgaagcccatgattagacacaagatggtatcagggcac
ttgctgctttgaataaatgtcagtctcctgtcttggaagaatgacctgacagggtaaagaggaacttgcagctgagaaaggct
ttagtgactcaagagctgaataattccccaaaagctggagcatcctggcatttccagctccccatctctgcttgttccacttcct
tggggctacatcaccatctacatcatcatcactcttccactccctcccttagtgccaactatgtttatagcgagatattttctgctc
attggggatcggaaggaagtgctgtggcctgagcggtctccttgggaagacaggatctgatacatacgttgcacaacctatt
tgacataagaggtttcacttcctgagatggatgggatggtagcagatttgggtccaggttacagggccaggatgagacatg
gcagaactgtggagactgttacgtcagggggcattgccccatggctccaaaatttccctcgagcgaaagcatcaggggct
catgcaacctggatactagtgctgcttcaaccacactgtgctattggatgagtcacttccaccctcctagccttgatttcttcgtc
tgctgttcacattcaaatagctattcatgtcttcatctctgtggtcccaccatatcccaccagacaatcattagggctcctcttag
ctggcagattctgaggtcctggatgctacaattggaagatggagaagtagaagctcaaggtttctgacctgtatcccaagtc
ccagaagcagaatggactaactcagagctgatgctcgggtcccttgcatatctcccttcctgtcactggctttgatcctccttc
gttcagcttgtaatcacatcaacagaccaaagacatctctgtgttctgtcaggagagttcacagagccaccaaccctccaga
ccctgctggttgccgcataaagactctgaggaagggtttgaggctgctgtgatcatgcaatgaatgcatgattgtaccactgc
actccagcctgggggataaaggtagatcctgtctaggagagagagagagagaaagagaaagagagagagaagggagg
gagagacaaagaaaaagagagagagggagggagaaagaaagagagaaagaaaagagaaaagaaagaaaaagaaa
gaaagagagagagggagggagggagagagaaagaaagaaagaaagagaaagagagaaagagagaaagagaaaga
aaggaagaaagaaagaaagaaagaaagaaagaaagaaagaaagaaagaaaagaaaagaaagaaagagagagagaa
agaaaaagaaagaggaaggaaggaaggaaggaagaaagacaggctctgaggaaggtggcagttcctacaacgggag
aaccagtggttaatttgcaaagtggatcctgtggaggcaaaacagaggagtcccctaggccacccagacagggcttttagc
tatctgcaggaccagacaccaaatttcaggagggctcagtgttaggaatggattatggcttatcaaattcacaggaaactaac
atgttgaacagcttttagatttcctgtggaaaatataacttactaaagatggagttcttgtgactgactcctgatatcaagatactg
ggagccaaattaaaaatcagaaggctgcttggagagcaagtccatgaaatgctctttttcccacagtagaacctatttccctc
gtgtctcaaatacttgcacagaggctcactcccttggataatgcagagcgagcacgatacctggcacatactaatttgaataa
aaatgctgtcaaattcccattcacccattcaagcagcaaactctaccacctgaatgtacatgccaggcactgtgctagacttg
gctcaaaaagatttcagtttcctggaggaaccaggaggagcaaggtttcaactcagtgctataagaagtgttacaggctgga
cacggtggctcacgcctgtaatcccaacactttgggaggccgaggcgggcagatcacaaggtcaggagatcgagaccat
cctggctaacatggtgaaaccctgtctctactaaaaatacaaaaaattagccgggcgtggcggcaggtgcctgtagtccca
gctgctggggaggctgaggcaggagaatggtgtgaacccgggaggcggaacttgcagggggccgagatcgtgccact
gcactccagcctgggcgacagagtgagactctgtctcaaaaaaaaaaaaaaagtgttatgatgcagacctgtcaaagagg
caaaggagggtgttcctacactccaggcactgttcataacctggactctcattcattctacaaatggagggctcccctgggca
gtaccctggagcaggcactttgctggtgtctcggttaaagagaaactgataactcttggttggtattaccaagagatagagtct
cagatggatattcttacagaaacaatattccacttttcagagttcaccaaaaaatcattttaggcagagctcatctggcattgatc
tggttcatccatgagattggctagggtaacagcacctggtcttgcagggttgtgtgagcttatctccagggttgccccaactc
cgtcaggagcctgaaccctgcataccgtatgttctctgccccagccaagaaaggtcaattttctcctcagaggctcctgcaat
tgacagagagctcctgaggcagagaacagcacccaaggtagagacccacaccctcaatacagacagggagggctattg
gcccttcattgtacccatttatccatctgtaagtgggaagattcctaaacttaagtacaaagaagtgaatgaagaaaagtatgt
gcatgtataaatctgtgtgtcttccactttgtcccacatatactaaatttaaacattcttctaacgtgggaaaatccagtattttaat
gtggacatcaactgcacaacgattgtcaggaaaacaatgcatatttgcatggtgatacatttgcaaaatgtgtcatagtttgcta
ctccttgcccttccatgaaccagagaattatctcagtttattagtcccctcccctaagaagcttcc
SEQ ID NO: 17
IGFBP3 promoter sequence
gtctattggtggtcagtaagactgatagccacaccccagaggacactgagaaacagtagtgctgtcagaaggagagagac
cctagggtggcttgtccagctgggggctgagcagaagaaaagcaaaggagccagtatcacgcagcgacaaggtgccctt
tagcagcaggagtgggctcaaagaaatgagtggtttaggtcacctggtgaaaataacgaggctgaccaaaggaaacacta
attataatcaggtcagctggactccgttagtgaaaagagatctatgcagcctcctgacctctattcggatggttattcgctacttt
tgcaatgattagcaattgtatgccaagcaccttcacagggggtaaacatctttctcactaaatactagtttatgttaaaaactaca
aacaccagatgaattggaagtttaacagaattaacgagcatgtcacaaaattaagttttatccagtccaaatccttcctgcaac
aattatgttccgggaggggcagggagcttgcagttatcaaataaaactggctaaataacaaaacacaattctccccgtgtca
ctgcctcaaatttactctgctgttgcaagatgtcatgataaaggtgatggcaatctatataaaacattgttattggaatagcctttg
aaagttgaaatgtgacttagtctttacgtgtgtcccgtggtccctgaggccgggctggacgcctggagctttggcgccccct
agccattcggcactgaacaagtttctccaactgcaatgcgagggaaaaaagaagagaaccaagaataaaacaaattttttaa
tcctttccaaaaagacattgtttctgatcttcccctgtccactctccagatgataaagctgaataaaaagtgctacactaaccag
tggtcagtccaggctctgctgcaacattgcactaaccagaactgcagacctgggacctcaagaattgcatttgatgccgaac
ccagctctaatttcagagtcaaggtctctgcgagtatttaaggaacggatgtaaacctgggggattcgttttgtttccttcaatttt
ccaatgaaatcagagatcctgttcttgggtgtcaacgcagatactagaaggaggtgatacaagagaaaggaaacagcaag
cgacgattatggcacggtttcctgtaaacaaggttgagtgtagccacagcctgagcactgtgggagaagagctcataagaa
aatgacggtgctgggccttcgtcaccccggggcactccattgttcttgtctttggtctctttttatttgtagaggtccaattatttat
ttatttagtacaagagggaacgaaattgatctttccattctaaaaggagagtatatatgtataaaaggaagctgtatagatatgg
gggaagaggtggacagggggaaaaggggagaggacgagagagagaaagggagggagagggacaaggagagaca
ctgggcgagagatcgattaggagagacagaaatgatgaatgaagattaacttcacccaaggcttcgtgctggaggggaat
ggaggagctcctgatttgctattactactccaaactgcaaagggctccttcaagtcacctatccacctcctaaggcaagcgtc
caatttcaacagcgttcaggaaagtctcctcccgcggaggtctcaccgcttcccactccacccccacaaactctttggaaaa
gtgccttgaaaaatttaatcctcaatccaatcctggaccaccagcgtcctctgttggtcaccgaagggagggggtgcgcag
acaaaactgaagaaactcgagtgccagagaaggccgacaggagttacagcgacctcagcgcgcaattgcgcccccgaa
ctttactgaaaagtgtttagattgcagagataagctagaatcccaacgcatcgagaatacagtaatacgaagtcgccttcaaa
aaatgacaatgaaaattgcctattaaaggactatttggttaattacgtttcagcagtgcccagtttattgtattattattcttttgtca
tgggtgtaaactccatttgaaaacataatcagggagaatacccaagacaagaagaacagttgtcatttaaaatatttgaaaag
ccctgccttaaggacgcattcgcttgccggtccactcttaattggagacttgcggtgtagcaacacgtgagagtcttcttgcgt
tgagaagtaagcctggaaaggggcgaaggccccgggcgcatcttcagatgcgtatttgtgggcccctggggatataaaca
gcccagcgggtgtaaattaaaccccgcagtgccttggctccctgagacccaaatgtaagtcagaaatgtcccaagacttcg
cctgccaacggaattaaattttagaaagctccacgaggtacacacgaatgcggagcgctgtatgccagtttccccgacacc
ggctcgccgcagggagacctcaccccgagagcggaaggggtaagggcggcggggtcaaggagatcgggggtgctga
gttggccaggagtgactggggtgaccgggggtgctgaggtggcctggagtgccggggtggccgggcacaccttggttct
tgtagacgacaaggtgacccgggctccgggcgtgcgcacgaggagcaggtgcccgggcgagtctcgagctgcacgcc
cccgagctcggccccggctgctcagggcgaagcacgggcccccgcagccgtgcctgcgccgacccgcccccctccca
acccccactcctgggcgcgccgttccggggcgtgtcctgggccaccccggcttctatatagcggccggcgcgcccgggc
cgcccagatgcgagcactgcggctgggcgctgaggatcagccgcttcctgcctggattccacagcttcgcgccgtgtact
gtcgccccatccctgcgcgcccagcctgccaagcagcgtgccccggttgcaggcgtca

The disclosures of each and every patent, patent application, and publication cited herein are hereby incorporated herein by reference in their entirety. While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising a disease-responsive reporter construct, comprising at least one promoter of at least one disease-responsive gene operably linked to at least one sequence encoding a reporter molecule.

2. The composition of claim 1, wherein the promoter of a disease-responsive gene comprises at least 250 nucleotides immediately upstream of a disease-responsive gene start codon.

3. The composition of claim 1, wherein the disease is the recurrence of a nephrotic syndrome (RNS), and further wherein the RNS-responsive gene is selected from the group consisting of ABCC6, AC005682.5, AC084018.1, ADAMTS1, ADAMTS5, ASB2, BAAT, BMF, CCDC147, CCL2, CDKN1C, CDKN2B, CHAC1, CSAD, CSGALNACT1, CYP1B1, CYP24A1, DDIT3, DTX3, FBXLS, GREM1, GSDMB, HCPS, HLA-DMB, HSD17B7P2, IGFBP3, IGFBP5, IL-11, IL-1A, IL-1β, KCTD19, KCTD4, KRT23, KRTAP1-1, KRTAP2-3, LINC00263, MAP3K7CL, NEAT1, NPPB, NR1D2, PLA2G4C, PLK1S1, PTGIS, RAPGEF3, RNF207, RP11-16112.1, RP11-336N8.4, RP5-1021120.2, RPL13AP20, RRNAD1, RUSC1-AS1, SEMA3A, SLC22A3, SLC25A27, SLC7A14, SMOC1, SMPD3, SUSD4, TMEM86A, TNF, TNFSF10, TRIM45, TRPC4, WDR66, XXbac-BPG246D15.8, TMEM2, KIAA0247, RERE, PDXK, TAP1, CARHSP1, NEDD9, TAPSAR1, CPD, CNDP2 and any combination thereof.

4. The composition of claim 3, wherein the RNS-responsive gene is selected from the group consisting of IGFBP3, IL-1β, BMF and any combination thereof.

5. The composition of claim 4, wherein the promoter is selected from the group consisting of SEQ ID NO:15, SEQ ID NO:16, SEQ ID NO:17, a nucleotide sequence having at least 90% identity to SEQ ID NO:15, a nucleotide sequence having at least 90% identity to SEQ ID NO:16, a nucleotide sequence having at least 90% identity to SEQ ID NO:17, a fragment comprising at least 60% of the full length of SEQ ID NO:15, a fragment comprising at least 60% of the full length of SEQ ID NO:16, a fragment comprising at least 60% of the full length of SEQ ID NO:17, and any combination thereof.

6. The composition of claim 1, wherein the reporter molecule is luciferase.

7. The composition of claim 1, wherein the reporter construct is selected from the group consisting of:
a) integrated into the genome of a cell line; and
b) on a plasmid.

8. A cell comprising a disease-responsive reporter construct of claim 1.

9. A method of diagnosing a disease or disorder in a subject in need thereof, the method comprising:
a) obtaining a test sample of the subject,
b) contacting a cell comprising at least one disease-responsive reporter construct of claim 1 with the test sample,
c) measuring the expression level of at least one reporter molecule,
d) comparing the expression level of at least one reporter molecule to the level of a comparator control, and
e) diagnosing the subject as having or at risk of the disease or disorder to which the disease-responsive reporter construct is responsive when the level of expression of at least one reporter construct is altered as compared to a comparator control.

10. The method of claim 9 wherein the promoter of a disease-responsive gene comprises at least 250 nucleotides immediately upstream of a disease-responsive gene start codon.

11. The method of claim 10, wherein the disease is RNS, and further wherein the RNS-responsive gene is selected from the group consisting of ABCC6, AC005682.5, AC084018.1, ADAMTS1, ADAMTS5, ASB2, BAAT, BMF, CCDC147, CCL2, CDKN1C, CDKN2B, CHAC1, CSAD, CSGALNACT1, CYP1B1, CYP24A1, DDIT3, DTX3, FBXLS, GREM1, GSDMB, HCPS, HLA-DMB, HSD17B7P2, IGFBP3, IGFBP5, IL-11, IL-1A, IL-1β, KCTD19, KCTD4, KRT23, KRTAP1-1, KRTAP2-3, LINC00263, MAP3K7CL, NEAT1, NPPB, NR1D2, PLA2G4C, PLK1S1, PTGIS, RAPGEF3, RNF207, RP11-16112.1, RP11-336N8.4, RP5-1021120.2, RPL13AP20, RRNAD1, RUSC1-AS1, SEMA3A, SLC22A3, SLC25A27, SLC7A14, SMOC1, SMPD3, SUSD4, TMEM86A, TNF, TNFSF10, TRIM45, TRPC4, WDR66, XXbac-BPG246D15.8, TMEM2, KIAA0247, RERE, PDXK, TAP1, CARHSP1, NEDD9, TAPSAR1, CPD, CNDP2 and any combination thereof.

12. The method of claim 11, wherein the RNS-responsive gene is selected from the group consisting of IGFBP3, IL-1β, BMF, and a combination thereof.

13. The method of claim 12, wherein the promoter is selected from the group consisting of SEQ ID NO:15, SEQ ID NO:16, SEQ ID NO:17, a nucleotide sequence having at least 90% identity to SEQ ID NO:15, a nucleotide sequence having at least 90% identity to SEQ ID NO:16, a nucleotide sequence having at least 90% identity to SEQ ID NO:17, a fragment comprising at least 60% of the full length of SEQ ID NO:15, a fragment comprising at least 60% of the full length of SEQ ID NO:16, a fragment comprising at least 60% of the full length of SEQ ID NO:17, and any combination thereof.

14. The method of claim 9, wherein the comparator control is at least one selected from the group consisting of: a positive control, a negative control, a historical control, a historical norm, or the level of a reference molecule in the biological sample.

15. The method of claim 9, wherein the subject is human.

16. The method of claim 11, wherein the RNS is recurrent focal and segmental glomerulosclerosis (rFSGS).

17. The method of claim 16, further comprising at least one step selected from the group consisting of administering a treatment for rFSGS to the subject.

18. The method of claim 16, wherein the method comprises differentially diagnosing a subject as having rFSGS.

19. A method of identifying a disease-responsive reporter gene, comprising:
a) contacting cells with a biological sample from a subject having a disease or disorder for which a disease-responsive reporter construct is desired;
b) isolating RNA from the cells; and
c) performing an analysis on the isolated RNA to identify candidate genes whose expression level was altered in the presence of the biological sample from the subject having the disease or disorder.

20. A method of constructing a disease-responsive reporter comprising:
a) identifying the promoter region of one or more disease-responsive reporter gene; and
b) generating a disease-specific reporter construct, wherein the reporter construct comprises a nucleotide sequence comprising the promoter of the candidate gene operably linked to a nucleotide sequence encoding one or more reporter markers.
